(19) United States
(12) Patent Application Publication
(10) Pub. No.: US 2024/0425751 A1
(43) Pub. Date: Dec. 26, 2024

(54) METHOD FOR PRODUCING ELECTROCHEMILUMINESCENCE NANOPROBE, ELECTROCHEMILUMINESCENCE NANOPROBE, ELECTROCHEMILUMINESCENCE SENSOR, ELECTROCHEMILUMINESCENCE DETECTION METHOD, AND KIT FOR ELECTROCHEMILUMINESCENCE DETECTION

(71) Applicants: CANON MEDICAL SYSTEMS CORPORATION, Tochigi (JP); Nanjing University, Nanjing (CN)

(72) Inventors: Xiaotian WU, Beijing (CN); Qiqi XU, Beijing (CN); Hao HUANG, Beijing (CN); Chao WANG, Nanjing (CN); Huangxian JU, Nanjing (CN); Jie WU, Nanjing (CN)

(73) Assignees: CANON MEDICAL SYSTEMS CORPORATION, Tochigi (JP); Nanjing University, Nanjing (CN)

(21) Appl. No.: 18/738,909

(22) Filed: Jun. 10, 2024

(30) Foreign Application Priority Data
Jun. 9, 2023 (CN) 202310682568.5

(51) Int. Cl. C09K 11/07; C08F 8/34; C12Q 1/34; G01N 21/76

(52) U.S. Cl. CPC C09K 11/07; C08F 8/34; C12Q 1/34; G01N 21/76; C09K 2211/1466; C09K 2211/1483

(57) ABSTRACT

A method for producing an electrochemiluminescence nanoprobe according to an embodiment includes: a hot exciton nanoparticle synthesis step of polymerizing a hot exciton organic luminescent molecule and a copolymer molecule to synthesize hot exciton nanoparticles; and a hot exciton nanoparticle modification step of modifying the obtained hot exciton nanoparticles with an oligonucleotide chain modified with a quencher molecule to obtain modified hot exciton nanoparticles.

### REFERENCE TO A SEQUENCE LISTING

In accordance with 37 CFR § 1.831-1.835 and 37 CFR § 1.77(b) (5), the specification makes reference to a Sequence Listing submitted electronically as a .xml file named “553524US_ST26”. This .xml file was generated on Jun. 10, 2024 and is 10,178 bytes in size. The entire contents of the Sequence Listing are hereby incorporated by reference.

### CROSS-REFERENCE TO RELATED APPLICATIONS

This application is based upon and claims the benefit of priority from Chinese Patent Application No. 202310682568.5, filed on Jun. 9, 2023, the entire contents of all of which are incorporated herein by reference.

### FIELD

Embodiments described herein relate generally to a method for producing an electrochemiluminescence nanoprobe, the electrochemiluminescence nanoprobe, an electrochemiluminescence sensor, an electrochemiluminescence detection method, and a kit for electrochemiluminescence detection.

### BACKGROUND

Electroluminescence (EL) is a phenomenon in which a luminescent material emits light under current and voltage excitation owing to an electric field and also a process of directly converting electrical energy into light energy. Organic electroluminescence is a phenomenon in which a thin-film element produced from an organic luminescent material emits light under electric field excitation. An organic electroluminescent element has a luminescence principle that, when driven by an external voltage, an electron injected from an electrode and a hole combine and release energy in an organic material, and the energy is transferred to a molecule of an organic luminescent substance to excite the molecule, so that a transition from a ground state to an excited state thus occurs, and, when the excited molecule returns from the excited state to the ground state, a radiative transition occurs to generate a luminescence phenomenon. Excited states can be classified into singlet (S) and triplet (T) excitons in accordance with spin multiplicity. An excited molecule may be in a lowest energy singlet excited state S₁ or triplet excited state T₁ or may be in a higher energy singlet excited state (S₂, S₃, S₄, . . . ) or triplet excited state (T₂, T₃, T₄, . . . ). When a radiative transition causes a molecule in the S₁ excited state to return to the S₀ ground state, the molecule generates “fluorescence emission”. When a radiative transition causes a molecule in the T₁ excited state to return to the S₀ ground state, the molecule generates “phosphorescent emission” (see FIG. 19). FIG. 19 is a schematic diagram illustrating the luminescence principle of a hot exciton material having hybrid locally-excited and charge-transfer excited state (HLCT) properties.

The electrochemiluminescence (ECL) technology is a method for generating specific luminescence by performing an electrochemical reaction in an electrode surface by the use of an electrochemical principle to generate an excited state. The excited state in the electrochemiluminescence process is generated by an electron transfer reaction between intermediate radicals formed by electron transfer between an electroactive substance and an electrode.

According to luminescent reagents, electrochemiluminescence-based materials can be classified into metal complex electrochemiluminescence-based materials and organic compound electrochemiluminescence-based materials (hereinafter, sometimes referred to as “organic luminescent materials”), for example, aromatic hydrocarbons such as organic fluorene, BODIPY derivatives, fluorene-based polymers, and various types of organic nanomaterials.

Examples of common electrochemiluminescent metal complexes include tris(bipyridine)ruthenium (II) complex ions (Ru(bpy)₃²⁺), which belong to phosphorescent emission-based materials. In the case of using the tris(bipyridine)ruthenium (II) complex ions as an ECL probe, exciton utilization can reach 100%, so that an electrochemiluminescence detection method based on tris(bipyridine)ruthenium (II) complex ions (Ru(bpy)₃²⁺) is a standard method for electrochemiluminescence detection. In contrast, the above-mentioned organic luminescent materials belong to fluorescent emission-based materials and exciton utilization thereof can achieve only 25%.

As excited states of organic luminescent materials having been currently used in the electrochemiluminescence field, there are mainly two conventional types of excited states, namely, a locally-excited (LE) state and a charge-transfer (CT) excited state. Due to electron-hole distributions in the two types of excited states and binding energy characteristics of resulting excitons, the utilization of excitons in the locally-excited state is low and radiative transition rate/quantum yield (Φ_{PL}) is high, or the utilization of excitons in the charge-transfer excited state is high and radiative transition rate/quantum yield is low. Thus, high utilization of both the excitons in the locally-excited state and the charge-transfer excited state and high radiative transition rate/quantum yield cannot be achieved at the same time, hence, luminescence efficiency and intensity cannot attain ideally optimal levels. This limits downstream applications, for example, the efficiency of electrochemiluminescence.

In 2012, Ma Eoguang and Yang Bing et al. innovatively applied a photophysical phenomenon of reverse intersystem crossing (RISC) to a design for an organic electroluminescent material and successfully developed a series of donor-acceptor fluorescent materials with different light colors. An effective high-level reverse intersystem crossing process greatly increases the exciton utilization of the electroluminescence element. Here, a conversion process between a higher triplet state (Tₙ, n≥2) and a singlet state (Sₘ, m≥1) is referred to as a hot exciton process, a luminescence mechanism realized based on the hot exciton process is referred to as a hot exciton mechanism, and a luminescent material with obvious hot exciton reverse intersystem crossing is referred to as a hot exciton material. The hot exciton material generally has a large Tₙ-T₁ energy level and a small Tₙ-Sₘ state energy level. The large Tₙ-T₁ energy level can suppress internal conversion from Tₙ to T₁, meanwhile the small Tₙ-Sₘ energy level can increase the rate of reverse intersystem crossing from Tₙ to Sₘ. The intersystem crossing from Tₙ to Sₘ and the internal conversion from Tₙ to T₁ mutually compete, hence, theoretically, the rate of reverse intersystem crossing can be sufficiently high, the internal conversion can be completely suppressed, high-level triplet excitons can be all converted into singlet excitons, and the utilization of excitons in a hot exciton material can reach 100% (see Yuwei Xu, “Study on high-level reverse intersystem crossing process and photoelectric performance of hot exciton luminescent material”, South China University of Technology, 2020, Ph.D. Thesis, and see FIG. 19). Thus, the hot exciton material can realize reverse intersystem crossing (hRISC) via an independent “hot exciton” channel to increase the rate of generation of a singlet S₁ exciton. At the same time, the S₁-state exciton exhibits high exciton utilization in the CT state and high radioluminescence characteristics in the LE state, hence the hot exciton material has hybrid locally-excited and charge-transfer excited state (HLCT) properties and can have high exciton utilization and high quantum yield at the same time. The hybrid locally-excited and charge-transfer excited state includes not only hybrid locally-excited (LE) state properties but also charge-transfer (CT) excited state properties, and is not a simple mixture of a hybrid locally-excited state and a charge-transfer excited state, but is a single excited state formed after hybridization.

Currently, the hot exciton material is mainly used in the field of semiconductor materials, for example, in the field of organic electroluminescent diodes (OLED) (see Xiaojie Chen, Dongyu Ma et al., “Hybridized Local and Charge-Transfer Excited-State Fluorophores through the Regulation of the Donor-Acceptor Torsional Angle For Highly Efficient Organic Light-Emitting Diodes”, CCS Chem. 2022, 4, 1284-1294), and is a novel luminescent material following the first generation organic luminescent material (fluorescent material), the second generation organic luminescent material (phosphorescent material), and the third generation organic luminescent material (thermally activated delayed fluorescence, TADF).

The hot exciton material has excellent optical properties and therefore has been desired to be applied to bio-detection. The specification of Chinese Patent Application Publication No. 110981821 describes an attempt to use the hot exciton material for fluorescence detection, but there has been no successful example of the use of the hot exciton material in the field of ECL detection yet.

### BRIEF DESCRIPTION OF THE DRAWINGS

FIG. 1 is a schematic diagram illustrating a method for producing an electrochemiluminescence nanoprobe according to the present invention;

FIG. 2 is a schematic diagram illustrating an ECL switch based on an ssDNA-BB NRs nanoprobe according to the present invention;

FIG. 3 is a schematic diagram illustrating the production of and detection by a sensor array based on ssDNA-BB NRs according to the present invention;

FIG. 4 is a schematic diagram contrastively illustrating the luminescence principle of a TADF compound and the luminescence principle of a hot exciton material;

FIG. 5A and FIG. 5B are diagrams illustrating results of nuclear magnetic (¹H NMR) and mass spectrometric analyses of a BCzP-BT molecule produced in Example 1 of the present invention;

FIG. 6A and FIG. 6B are diagrams illustrating analysis results of excited states of the BCzP-BT molecule produced in Example 1 of the present invention;

FIG. 7A and FIG. 7B are diagrams illustrating properties of the excited states of the BCzP-BT molecule produced in Example 1 of the present invention;

FIG. 8A and FIG. 8B are diagrams illustrating analysis results for possibilities of a hot exciton process of the BCzP-BT produced in Example 1 of the present invention;

FIG. 9A to FIG. 9E are diagrams illustrating analysis results of characteristics of the BB NRs produced in Example 1 of the present invention;

FIG. 10A and FIG. 10B are diagrams illustrating analysis results of ECL characteristics of the BB NRs produced in Example 1 of the present invention;

FIG. 11A to FIG. 11F are diagrams illustrating ECL analysis results of the BB NRs produced in Example 1 of the present invention, in the presence of a co-reactive agent;

FIG. 12A to FIG. 12D are diagrams illustrating the ECL efficiency of the BB NRs produced in Example 1 of the present invention, based on Ru(bpy)₃²⁺;

FIG. 13A to FIG. 13C are diagrams illustrating analysis results of ECL attribution of the BB NRs produced in Example 1 of the present invention;

FIG. 14A to FIG. 14C are diagrams illustrating analysis results of characteristics of an ssDNA-BB NRs probe produced in Example 2 of the present invention;

FIG. 15A and FIG. 15B are diagrams illustrating analysis results of the feasibility of a signal switch of the ssDNA-BB NRs probe produced in Example 2 of the present invention;

FIG. 16A to FIG. 16C are diagrams illustrating detection results of ECL detection performed under different detection conditions in Example 3 of the present invention;

FIG. 17A to FIG. 17C are diagrams illustrating results of ECL imaging using ITO and Au/ITO electrodes in Example 3 of the present invention;

FIG. 18A and FIG. 18B are diagrams illustrating results of ECL detection of an HPV16 DNA target nucleic acid in Example 3 of the present invention by using a sensor array; and

FIG. 19 is a schematic diagram illustrating the luminescence principle of a hot exciton material having hybrid locally-excited and charge-transfer excited state (HLCT) properties.

### DETAILED DESCRIPTION

A problem to be solved by the present invention is to provide a method for producing an electrochemiluminescence nanoprobe with a wide detection range and high sensitivity, the electrochemiluminescence nanoprobe, an electrochemiluminescence sensor, an electrochemiluminescence detection method, and a kit for electrochemiluminescence detection.

A method for producing an electrochemiluminescent nanoprobe according to an embodiment includes: a hot exciton nanoparticle synthesis step of polymerizing a hot exciton organic luminescent molecule and a copolymer molecule to synthesize hot exciton nanoparticles; and a hot exciton nanoparticle modification step of modifying the obtained hot exciton nanoparticles with an oligonucleotide chain modified with a quencher molecule to obtain modified hot exciton nanoparticles.

As is well known, an organic electroluminescent diode (OLED) is an all-solid element and includes no liquid component. In the organic electroluminescent diode, a luminescent molecule is excited under the action of an external voltage to make a transition from a ground state to an excited state, and, when the resulting excited molecule returns from the excited state to the ground state, radiative transition is caused to generate luminescence. On the other hand, an environment for detection of electrochemiluminescence is liquid, a luminescent molecule undergoes an electrochemical reaction in an electrode surface to generate an excited state and emits light through radiative transition. Therefore, the organic electroluminescent diode and electrochemiluminescence are different in luminescence principle and luminescence environment. In the case of using a hot exciton material currently used in the field of organic electroluminescent diodes (OLED) as a luminescent molecule for electrochemiluminescence (ECL) detection, problems such as feasibility and reliability are first considered.

The inventors have intensively studied the above-mentioned problem, and, as a result, the inventors found that, by producing an electrochemiluminescent nanoprobe (hereinafter, sometimes referred to as “the ECL nanoprobe”) from a hot exciton organic luminescent molecule by nanotechnology and modifying the electrochemiluminescent nanoprobe with an oligonucleotide chain modified with a quencher molecule, the overall efficacy of electrochemiluminescence can be enhanced, so that quick, non-amplified, high-throughput detection of a target molecule with a wide detection range and extremely high sensitivity can be achieved.

In the present invention, a hot exciton organic luminescent molecule is applied to the field of electrochemiluminescence for the first time and nanoparticles are produced using the hot excitonic organic luminescent molecule as a luminescent material for the first time, whereby quick, non-amplified, high-throughput detection for a target molecule can be achieved, and there can be provided an electrochemiluminescence (ECL) nanoprobe based on the hot exciton organic luminescent molecule with a wide detection range and extremely high sensitivity, and a sensor a detection method, and a detection kit that use the electrochemiluminescence nanoprobe.

Hereinafter, a specific embodiment of the present invention will be described in detail. Note that the following description about the embodiment is only to explain an inventive concept of the present invention and is not intended to limit the present invention.

### Electrochemiluminescence Nanoprobe and Method for Producing Same

One embodiment of the present invention relates to a method for producing an electrochemiluminescent nanoprobe, the method including: a hot exciton nanoparticle synthesis step of polymerizing a hot exciton organic luminescent molecule and a copolymer molecule to synthesize hot exciton nanoparticles; and a hot exciton nanoparticle modification step of modifying the obtained hot exciton nanoparticles with an oligonucleotide chain modified with a quencher molecule to obtain modified hot exciton nanoparticles.

### Hot Exciton Organic Luminescent Molecule

The hot exciton organic luminescent molecule to be used in the present invention is not limited to a particular one, and any hot exciton organic luminescent molecule having hybrid locally-excited and charge-transfer excited state properties can be used, and examples thereof include various types of hot exciton organic luminescent molecules described in the above-mentioned “Study on high-level reverse intersystem crossing process and photoelectric performance of hot exciton luminescent material” and “Hybridized Local and Charge-Transfer Excited-State Fluorophores through the Regulation of the Donor-Acceptor Torsional Angle For Highly Efficient Organic Light-Emitting Diodes”.

The hot exciton material is a molecule having hybrid locally-excited and charge-transfer (HLCT) excited state properties. Note that the molecule having the hybrid locally-excited and charge-transfer excited state properties is not necessarily the hot exciton material. For example, singlet and triplet materials have the hybrid locally-excited and charge-transfer excited state properties, but do not belong to the hot exciton material.

The hot exciton material commonly has a one-dimensional fused ring structure as a structural unit and includes a donor cell including a heteroatom.

Examples of the fused ring structure include a benzene ring, a naphthalene ring, and an anthracene ring. These benzene, naphthalene, and anthracene rings may have a substituent.

A specific configuration structure of a donor-acceptor unit may be, for example, a D-A or D-A-D structure. D denotes a donor unit (donor) and A denotes an acceptor unit (Acceptor).

Examples of the donor-acceptor unit include units composed of a donor (D) selected from the following group (a) consisting of chemical formulae (1) and an acceptor (A) selected from the following group (b) consisting of chemical formulae (2).

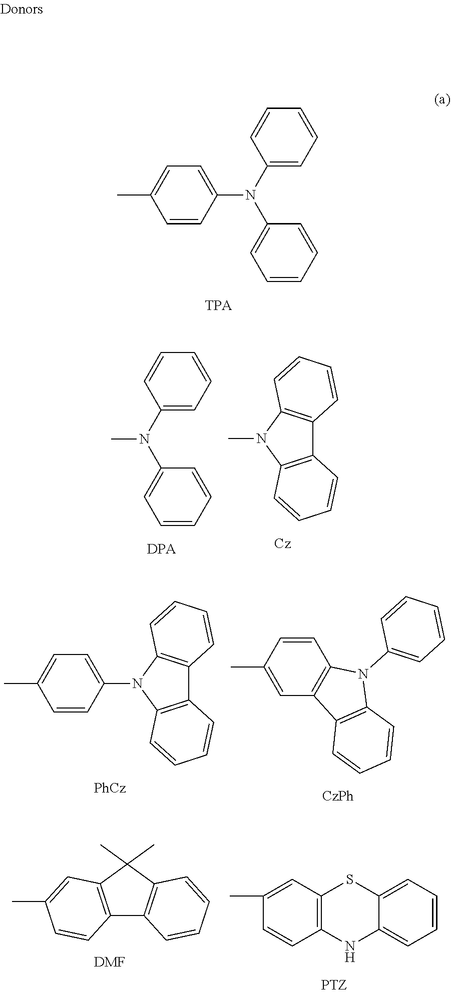

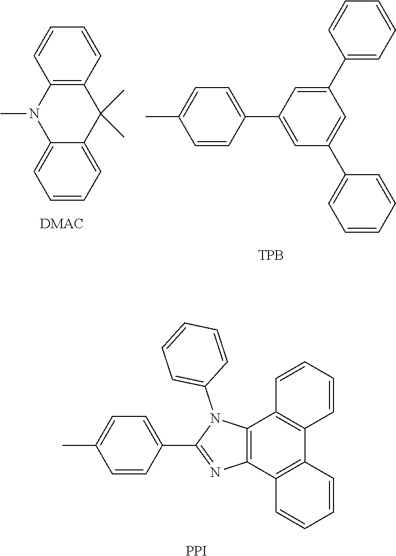

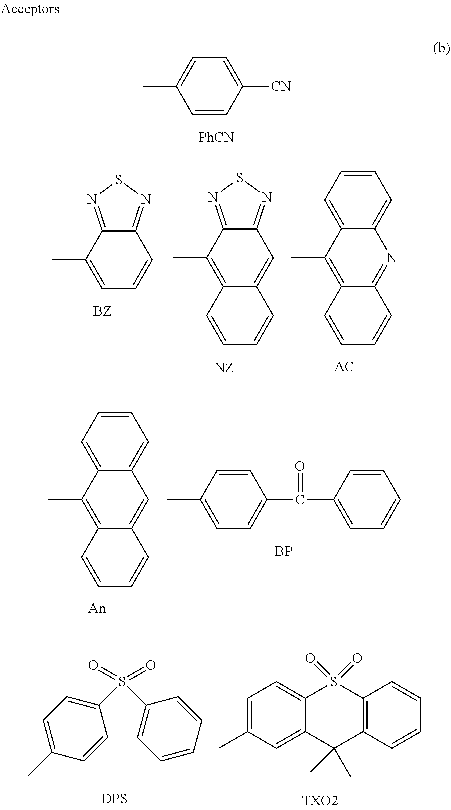

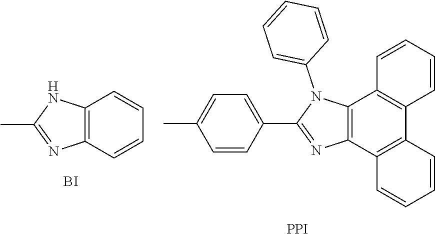

The hot exciton material is a sort of luminescent material breaking through the spin-statistic limit of conventional fluorescent materials, and includes blue-light hot exciton materials, green-light hot exciton materials, and red-light hot exciton materials in terms of the type of light emitted.

Since the donor-acceptor intensity of the hot exciton material is appropriate, pure-blue light emission and deep-blue light emission are more easily obtained in the molecular design of D-A fluorescent materials. Examples of a current successfully produced blue-light hot exciton material include a phenanthrimidazole unit-containing blue-light hot exciton material, an anthracene unit-containing blue-light hot exciton material, and other types of blue-light hot exciton materials.

Examples of the phenanthrimidazole unit-containing blue-light hot exciton material include phenanthrimidazole unit-containing blue-light hot exciton materials expressed by the following chemical formulae (3) and (4).

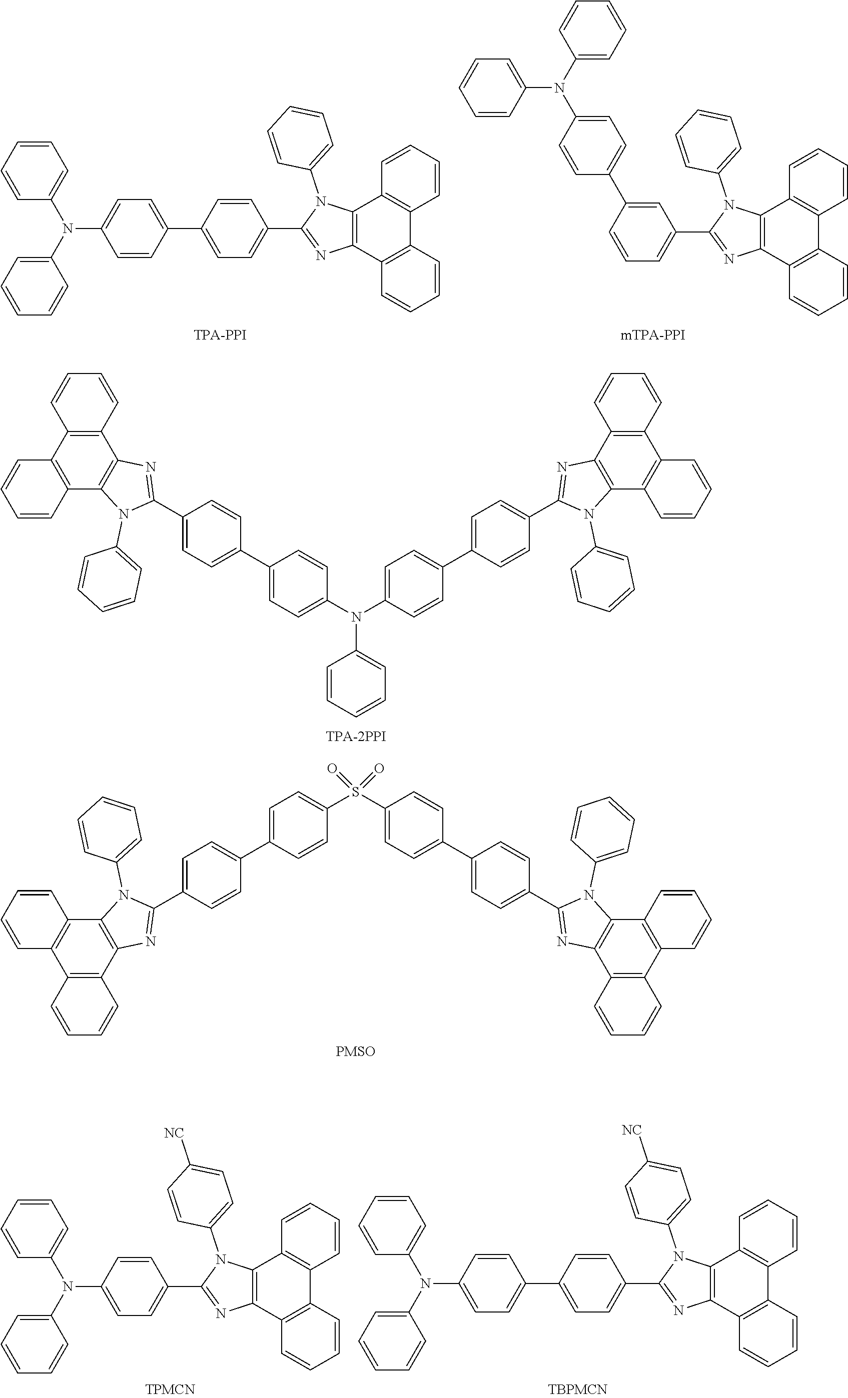

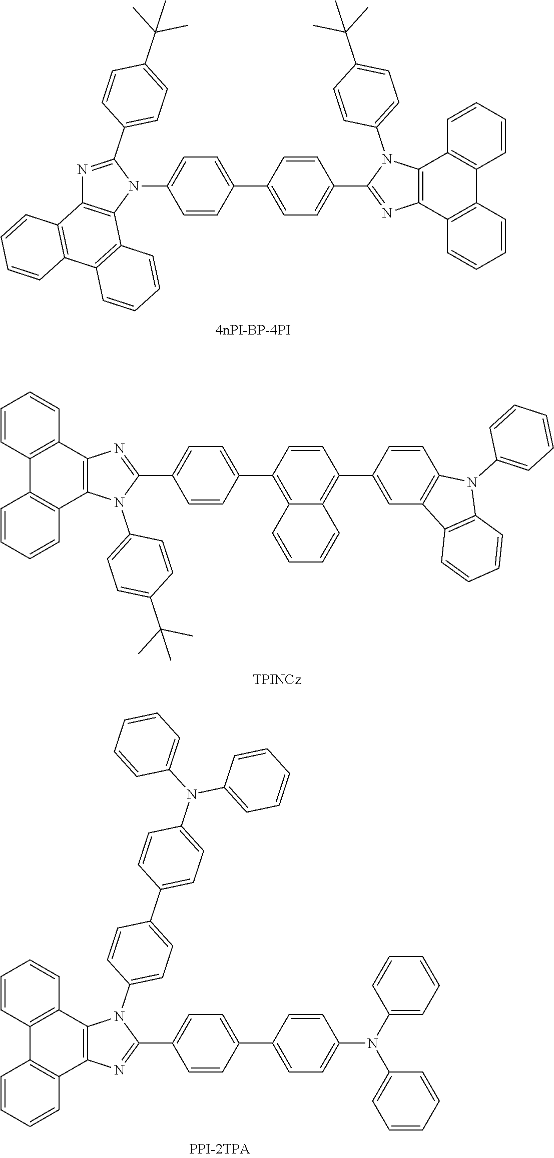 [SMILES: CC(C)(C)c1ccc(-c2nc3c4ccccc4c4ccccc4c3n2-c2ccc(-c3ccc(-c4nc5c6ccccc6c6ccccc6c5n4-c4ccc(C(C)(C)C)cc4)cc3)cc2)cc1 ; CC(C)(C)c1ccc(-n2c(-c3ccc(-c4ccc(-c5ccc6c(c5)c5ccccc5n6-c5ccccc5)c5ccccc45)cc3)nc3c4ccccc4c4ccccc4c32)cc1 ; c1ccc(N(c2ccccc2)c2ccc(-c3ccc(-c4nc5c6ccccc6c6ccccc6c5n4-c4ccc(-c5ccc(N(c6ccccc6)c6ccccc6)cc5)cc4)cc3)cc2)cc1]

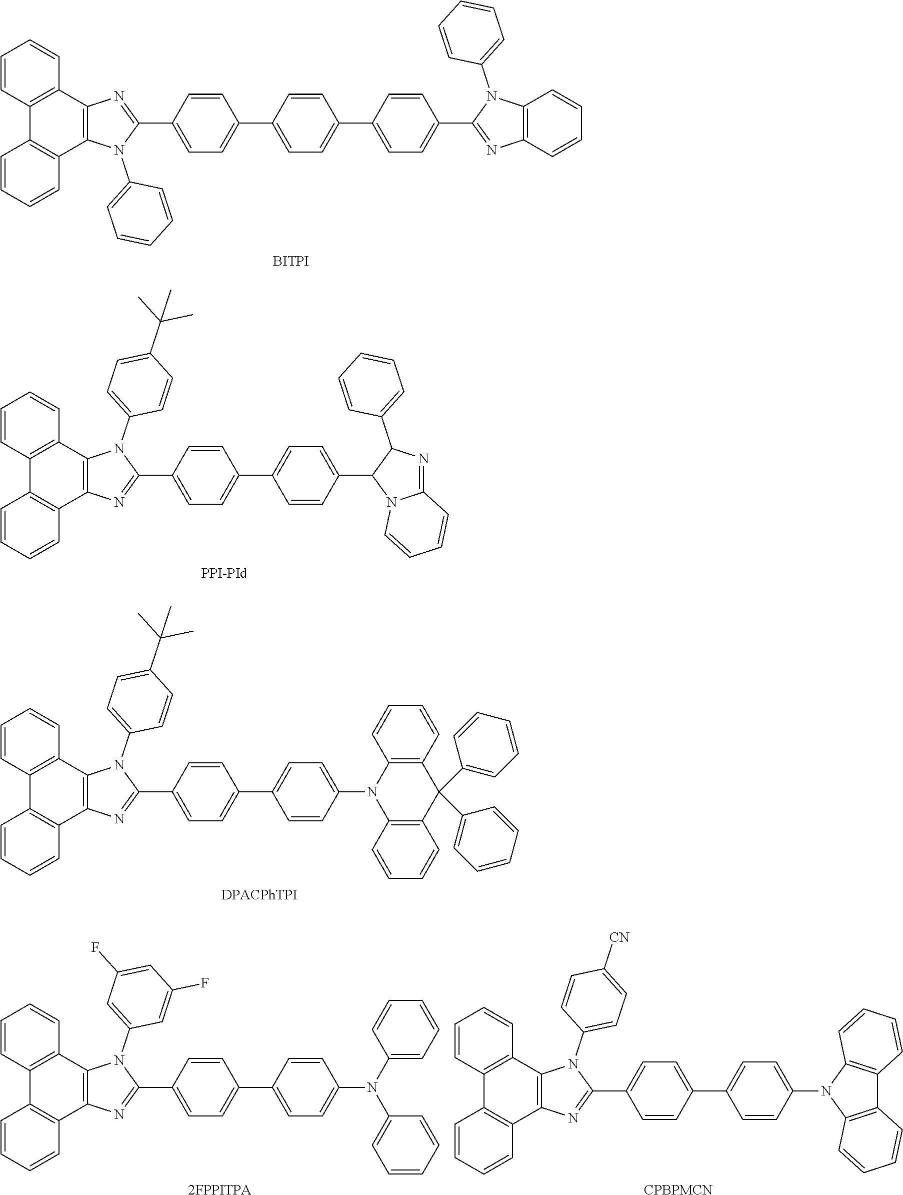

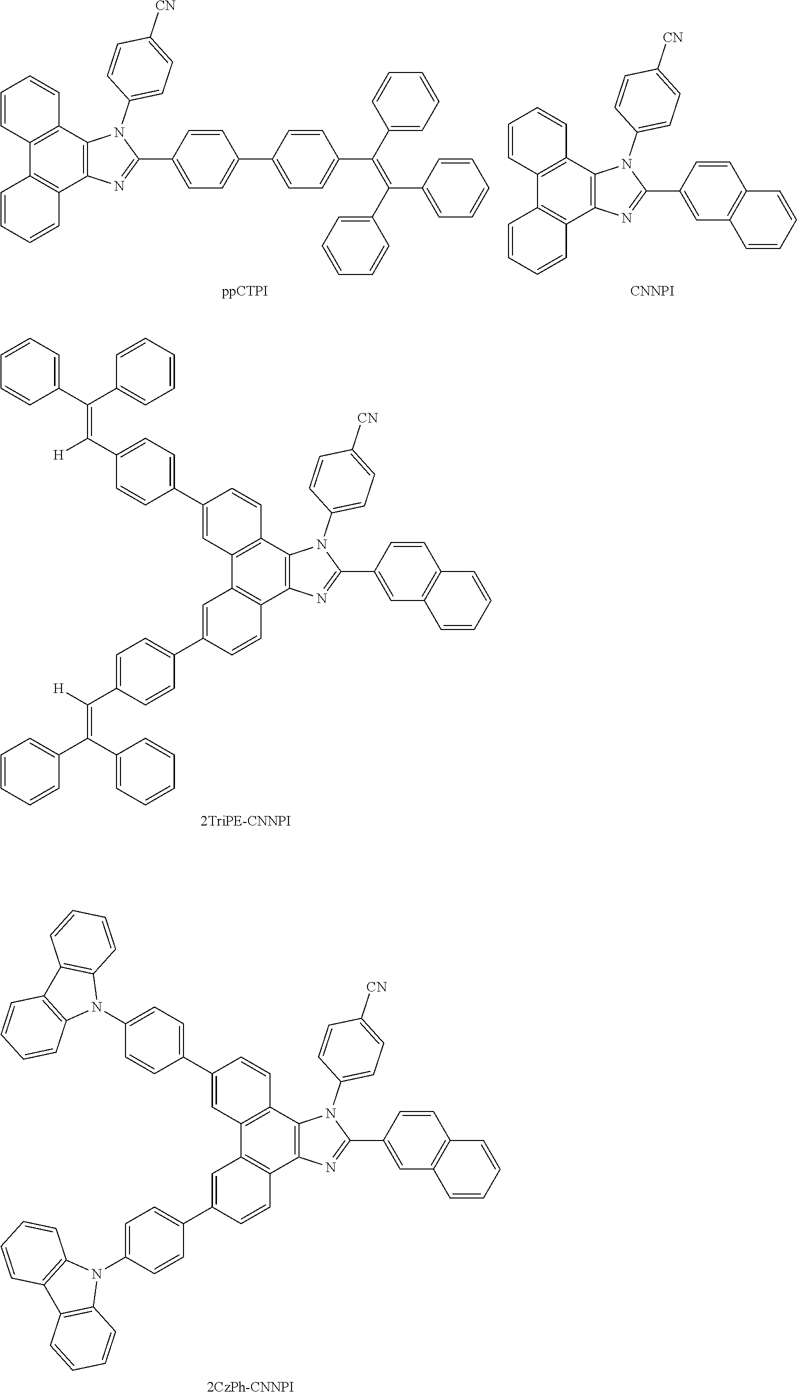

Examples of the anthracene unit-containing blue-light hot exciton material include anthracene unit-containing blue-light hot exciton materials expressed by the following chemical formulae 5.

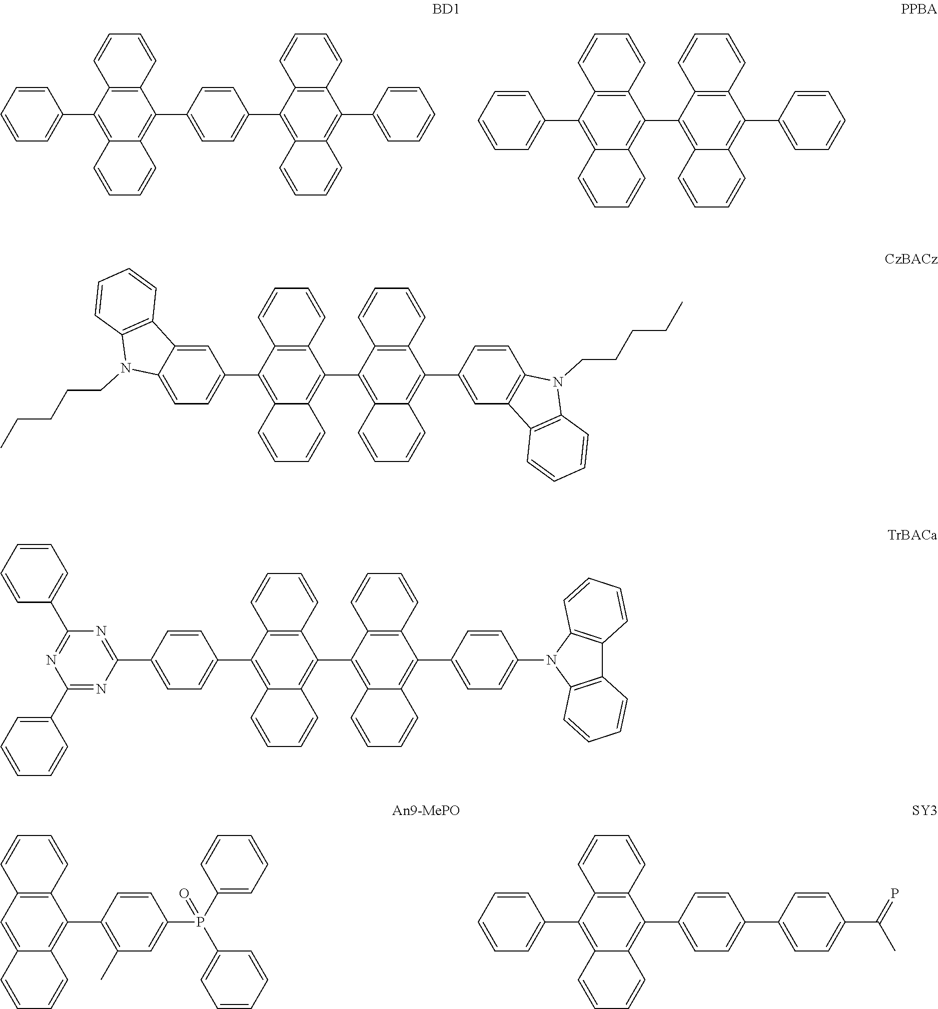

Examples of other types of the blue-light hot exciton materials include blue-light hot exciton materials expressed by the following chemical formulae 6.

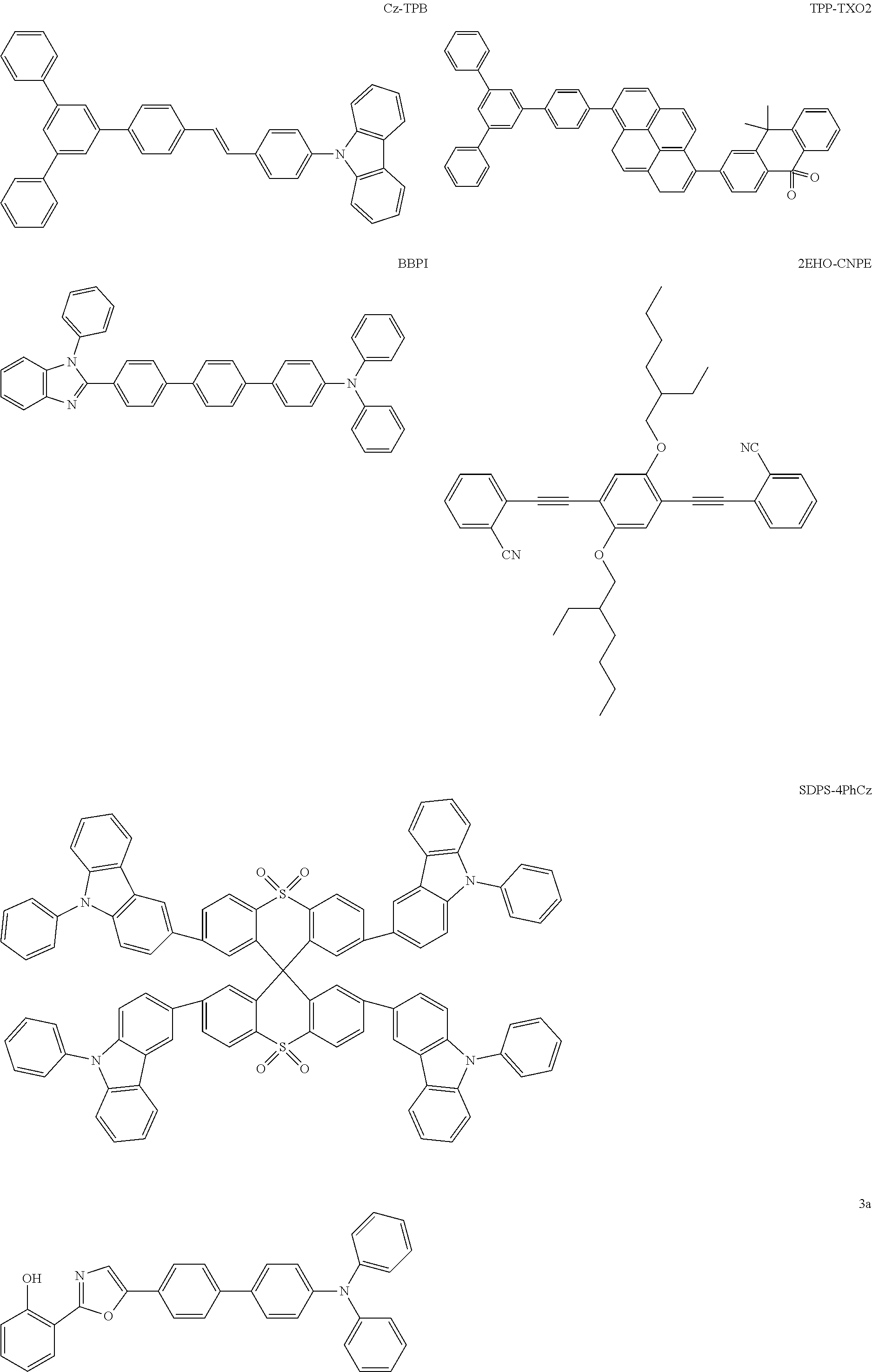

Typical examples of the green-light hot exciton material include green-light hot exciton materials expressed by the following chemical formulae 7.

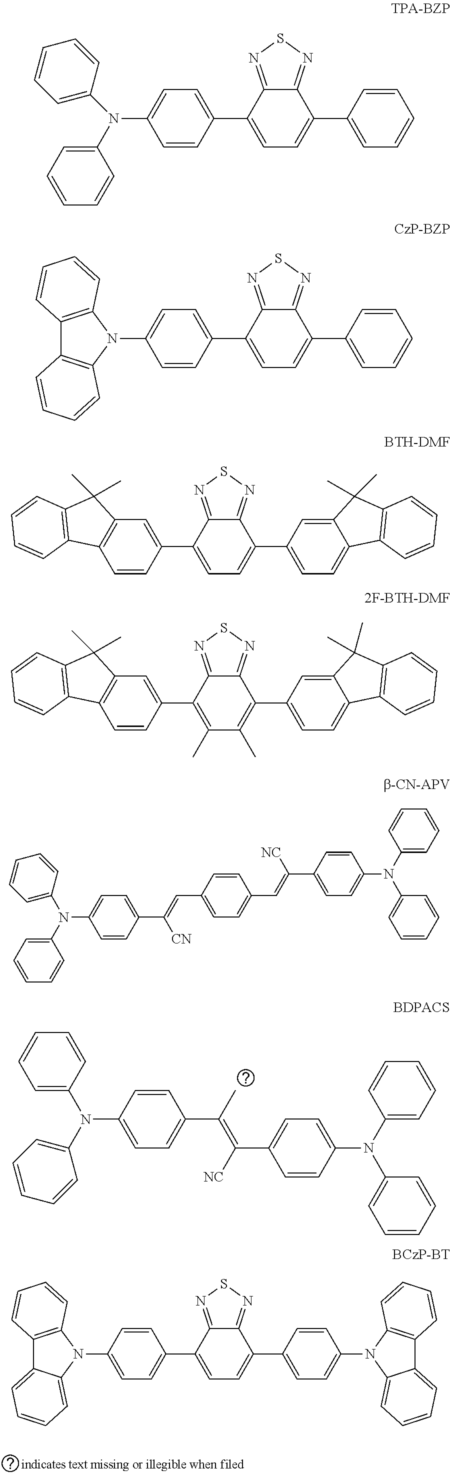

Typical examples of the red-light hot exciton material include red-light hot exciton materials expressed by the following chemical formulae 8.

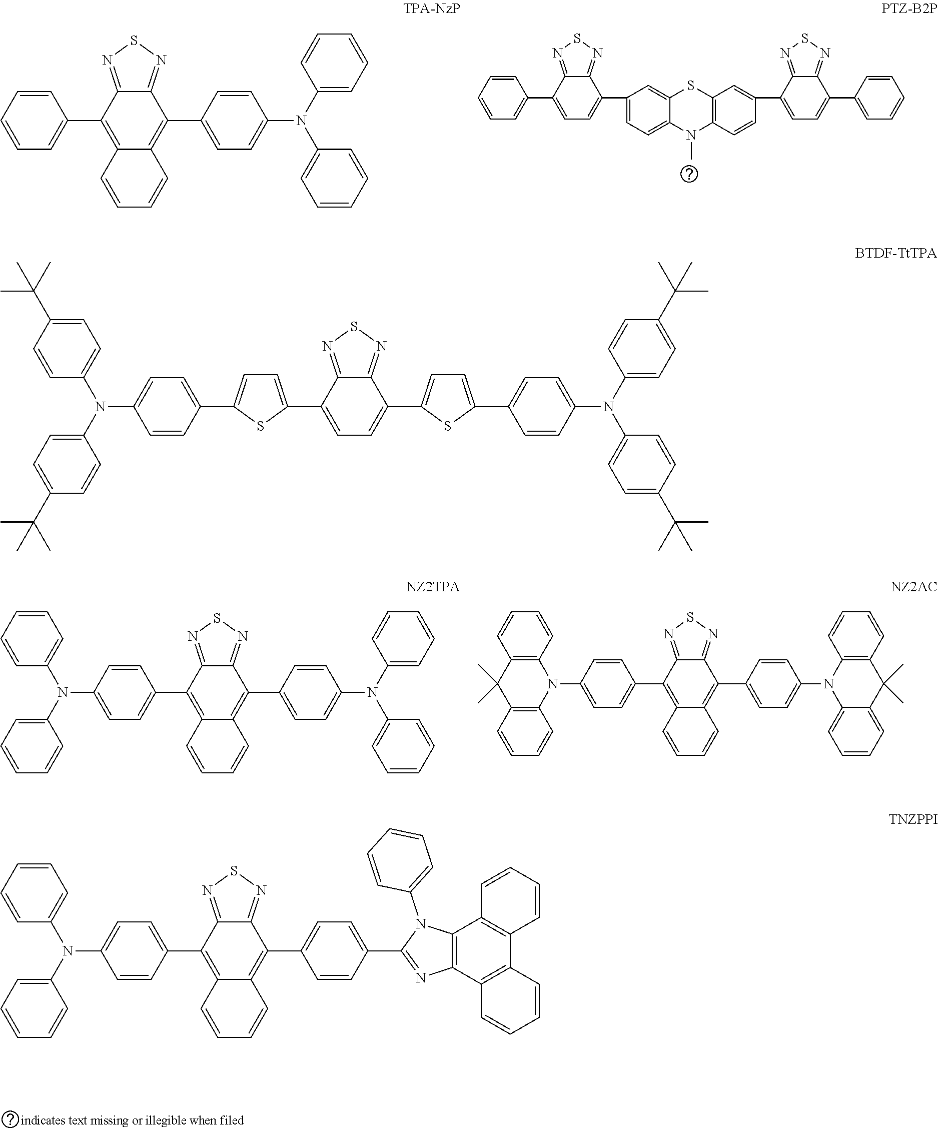

For the D-A-D hot exciton material, the binding of the acceptor (A) unit to the donor (D) unit may be ortho, meta, or para binding. Here, since spatial resistance can be reduced in the para binding, a twist angle between the acceptor (A) unit and the donor (D) unit decreases, a more locally excited component can be provided, and faster radiation-attenuation is achieved. The para binding is preferred because, according to circumstances, the para binding can achieve higher external quantum efficiency (EQE) than the ortho binding and the meta binding. For example, 4,7-bis(4-(9H-carbazol-9-yl)phenyl)benzo[c][1,2,5]thiadiazole (p-BCzP-BT) is illustrated in examples according to the present invention. In the above-mentioned compound, an electron donor (D) is carbazole and an electron acceptor (A) is benzothiadiazole.

Note that any hot exciton material having hybrid locally-excited and charge-transfer excited state (HLCT) properties can be used in the present invention. The hot exciton materials mentioned above are merely examples. As those skilled in the art will appreciate, derivatives formed by substituting the above-mentioned compound with one or more substituents (for example, an alkyl group, a hydroxy group, and halogen), and spatial isomers thereof, and the likes can be used as well in the present invention.

The method for producing the hot exciton material to be used in the present invention is not limited to a particular one. For example, the hot exciton material can be synthesized by a chemical reaction via a synthetic route of a Suzuki coupling reaction, a Scherrer coupling reaction, a cross-coupling reaction, or the like, or alternatively can be purchased through a commercial channel.

Although a synthetic route of the Suzuki coupling reaction is employed in the examples of the present invention, other synthetic routes may be employed as necessary. The Suzuki coupling reaction is a commonly used carbon-carbon bond formation reaction, in which zero-valent palladium is used as a catalyst and aryl or alkenylboronic acid is allowed to react with halogenated aromatic hydrocarbon such as chlorine, bromine, or iodine. Examples of the palladium catalyst that can be used include tetrakis(triphenylphosphine)palladium (Pd(PPh₃)₄), Pd[P(o-toly)₃]₃, and a Buchwald catalyst. As aryl or the alkenyl boronic acid and the halogenated aromatic hydrocarbon, their respective compound raw materials can be employed according to an electron donor (D) and an electron acceptor (A) in a hot exciton organic luminescent molecule synthesized as necessary, and are not limited to particular ones.

### Synthesis of Hot Exciton Nanoparticles

In the present invention, hot exciton nanoparticles are synthesized by polymerizing a hot exciton organic luminescent molecule and a copolymer molecule.

Nanoparticles in the present invention are particles within the nano-size range (0.1 to 100 nm) in at least one dimension in a three-dimensional space. Specifically, the nanoparticles may be in various geometric forms such as nanoballs, nanotubes, nanorods, and nano-onions. When a hot exciton organic luminescent molecule is formed in a nanoform, an ECL response signal can be effectively amplified and a detection limit, a detection range, selectivity, stability, and the like of an ECL sensor can be enhanced. In the present invention, a material in the form of nanorods (NR) is successfully produced as in the later-described examples, but, the nanoparticles that can be used in the present invention are not limited to nanorods, but can be formed in other nanoforms such as nanoballs can be formed, depending on the compound raw material and the synthetic route to be employed.

In the present invention, the hot exciton organic luminescent molecule and the copolymer molecule can be synthesized by a known method for producing an organic material nanostructure according to the conventional art, such as a nano-coprecipitation method, a microemulsion method, or a self-assembly method (Liu Ronghua, “Production and Application of Novel Fluorescent Conjugated Polymer Nanoparticles”, University of Science and Technology Beijing, 2018, Ph.D. Thesis), and the method is not limited to a particular one. Here, from the viewpoint of biocompatibility, the microemulsion method and the nano-coprecipitation method are more preferably used.

In the microemulsion method, a luminescent molecule is coated with a non-toxic, non-immunogenic, hydrophilic polymer, for example, a polyethylene glycol-based molecule such as polyethylene glycol (PEG) or polyethylene glycol-phosphatidylethanolamine (PEG-PE), or a biopolymer such as phospholipid, whereby luminescent molecular nanoparticles with high biocompatibility and high stability can be obtained.

In the nano-coprecipitation method, luminescent molecules are dissolved in a good solvent, a poor solvent is injected in large amounts under ultrasonic conditions, whereby the solubility of the luminescent molecules promptly decreases and the luminescent molecules aggregate and thereby precipitate in the form of small particles, and then the remaining organic solvent is removed to obtain luminescent molecular nanoparticles. The nano-coprecipitation method is preferably used because the method can be easily operated and the nanoparticles produced thereby are smaller and uniform in size.

In the nano-coprecipitation method, luminescent molecular nanoparticles can be surface-functionalized using an amphiphilic polymer. In a production process, the luminescent molecules and the amphiphilic polymer are self-organized, and the amphiphilic polymer has a hydrophobic functional group at one end thereof and has a hydrophilic functional group at the other end thereof, hence luminescent molecular nanoparticles coated with the amphiphilic polymer can be obtained and the functional groups (for example, an amino group and a carboxyl group) of the amphiphilic polymer are exposed to outer surfaces of the nanoparticles, and thus surface-functionalized luminescent nanoparticles are obtained. A functional group of a nucleic acid, polypeptide, sugar, protein, an antibody, or the like reacts with the functional groups of the amphiphilic polymer that are exposed to the outer surfaces of the nanoparticles by a condensation reaction or a bioorthogonal click reaction to obtain luminescent molecular nanoparticles having an identification function.

In the present invention, the amphiphilic polymer is also referred to as a copolymer molecule. As the copolymer molecule, copolymer molecules commonly used in the relevant field, such as a polystyrene-polyacrylic acid block copolymer (PS-PAA), a polystyrene-maleic anhydride copolymer (PSMA), and a poly(isobutylene-alt-maleic anhydride) (PIMA), which are expressed by the following chemical formulae 9, can be used, and the copolymer molecule is not limited to a particular one.

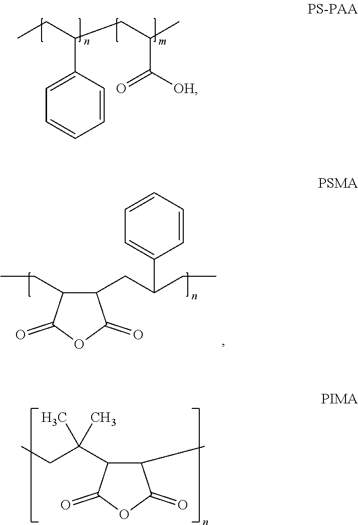

The diameter of the nanoparticles obtained by the nano-coprecipitation method can be 1 nm to 2 nm at minimum.

Note that the surface functionalization modification of hot exciton nanoparticles is not limited to the above-described modification method using the amphiphilic polymer, and other modification methods such as direct functionalization (in other words, directly modifying a luminescent molecule with an alkoxy chain, an amino/carboxyl group, or a functional group of a biomolecule or the like having a target function by a covalent-bonding method) and physical coating (in other words, coating nanoparticles with a phospholipid, silicon dioxide, or the like) can be employed.

The surface functionalization of luminescent molecular nanoparticles allows a large number of functional groups to be given to the surface of each nanoparticle, and furthermore allows a large number of oligonucleotides to be bonded, and thus an electrochemical signal can be amplified, so that the sensitivity of ECL detection can be enhanced.

### Modification of Hot Exciton Nanoparticles

In the present invention, the modification of hot exciton nanoparticles means oligonucleotide modification of hot exciton nanoparticles. Specifically, the above-mentioned surface-functionalized hot exciton nanoparticles are modified with an oligonucleotide chain modified with a quencher molecule to obtain oligonucleotide-modified hot exciton nanoparticles (hereinafter, sometimes referred to as “the ECL nanoprobe”).

A method for binding between oligonucleotide and the hot exciton nanoparticles is not limited to a covalent-bonding reaction (for example, cross-linking by NH₂—COOH), and may be other binding methods such as electrostatic action and affinity adsorption.

The oligonucleotide chain used for the modification may be a double-stranded oligonucleotide chain (for example, dsDNA) or may be a single-stranded oligonucleotide chain (for example, RNA or ssDNA). Specifically, the oligonucleotide chain can be suitably selected according to the type of a Cas enzyme to be used.

The collateral cleavage activity of the Cas enzyme is nonspecific, hence a nucleotide sequence constituting the oligonucleotide chain is not limited to a particular one and can be any sequence. The length of the oligonucleotide chain is also not particularly limited as long as a quencher molecule can be bonded to hot exciton nanoparticles and resonance energy can be transferred between the quencher molecule and the hot exciton nanoparticles, and can be designed with reference to various types of oligonucleotide chains for linkage that have been commonly used in the prior art.

From the viewpoint of cleavage sensitivity owing to the Cas enzyme, the number of nucleotides in the oligonucleotide chain as a single chain is better not too large and is preferably within a range of 3 to 20, more preferably within a range of 4 to 11, and still more preferably 5, 6, 7, 8, 9, 10, 11, or 12.

The oligonucleotide chain according to the present invention is modified with a quencher molecule. Dynamic quenching of the quencher molecule is caused by fluorescence resonance energy transfer (FRET) or collisional quenching. In the quencher molecule, optical absorption and the fluorescence emission of the hot exciton nanoparticles overlap, hence resonance energy can be transferred from the excited nanoparticles to the quencher molecule, whereby emission by the hot exciton organic luminescent molecule can be quenched.

The quencher molecule can be any quencher molecule having resonance energy transfer characteristics and being commonly used in the relevant field, and is not limited to a particular one. Examples of the quencher molecule that can be used include black hole quenchers (Black Hole Quencher™ reagents, that is, BHQ-based quenchers), dark quenchers (for example, DABCYL series quenchers), BlackBerry™ quenchers (that is, BBQ-series quenchers), Blueberry quenchers (that is, BLU-series quenchers), and amine reactive quenchers (QSY-series quenchers). Of these quenchers, the BHQ quenchers are often used. Any of these quenchers can be commercially obtained (for example, from LGC, BiochTechnologies™).

A method for binding of the quencher molecule to oligonucleotide is not limited to a particular one, and can be any method commonly used in the relevant field. The quencher molecule may be bound to an end of the oligonucleotide or may be inserted into an oligonucleotide sequence.

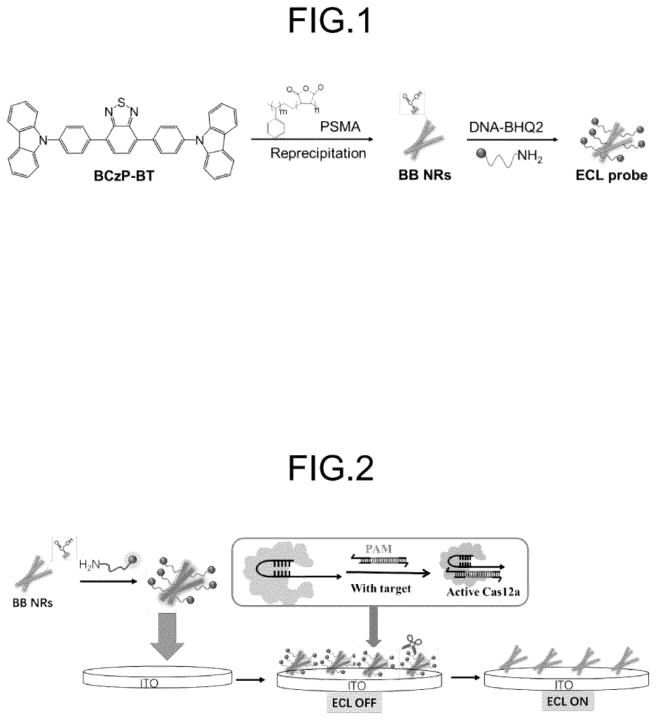

FIG. 1 is a schematic diagram illustrating a method for producing an electrochemiluminescence nanoprobe according to the present invention. A hot exciton organic luminescent molecule, namely, a BCzP-BT molecule produced in Example 1 of the present invention will be taken as an example. The hot exciton organic luminescent molecule, the BCzP-BT molecule, is polymerized with a copolymer molecule PSMA by the nano-coprecipitation method to obtain hot exciton nanoparticles (BB NRs). Then, oligonucleotide DNA bound to a quencher molecule BHQ at an end thereof is bound to hot exciton nanoparticles by a NH₂—COOH covalent-bonding reaction, whereby an electrochemiluminescent nanoprobe (ECL nanoprobe) of the present invention is obtained.

In the present invention, the oligonucleotide modified with the quencher molecule functions as a signal switch of the present invention together with a Cas enzyme described below. Specifically, when the cleavage of oligonucleotide by an activated Cas enzyme does not occur, the quencher molecule is bound to surfaces of the hot exciton nanoparticles via the oligonucleotide and exerts a quenching effect on the hot exciton organic luminescent molecule and a sensor electrode indicates a low ECL intensity (ECL OFF). In contrast, when the cleavage of oligonucleotide by the activated Cas enzyme occurs, the quencher molecule leaves the surfaces of the hot exciton nanoparticles, whereby the sensor electrode indicates a strong ECL signal (ECL ON).

### Electrochemiluminescence Sensor

An electrochemiluminescence sensor of the present invention (hereinafter, sometimes referred to as “the ECL sensor”) can be obtained by dropping the above-described ECL nanoprobe onto a working electrode.

The working electrode to be used in the present invention is not limited to a particular one and can be one of electrodes commonly used for ECL detection, such as a glass carbon electrode (GCE), an indium tin oxide (ITO) electrode, and a screen printed electrode (SPE). An indium tin oxide electrode is preferably used. A three-electrode system is more preferably used. In the electrode system, for example, a platinum wire counter electrode, an Ag/AgCl reference electrode, and an indium tin oxide (ITO) working electrode are arranged.

As the present invention is verified in the later-described examples, gold has a good catalytic effect on a coreactant tripropylamine or the like, hence a gold-indium tin oxide (Au-ITO) electrode can more greatly enhance an ECL signal than an ITO electrode, and therefore, an Au-ITO electrode is more preferably used in the present invention.

### Electrochemiluminescence Detection Method

An electrochemiluminescence detection method of the present invention (hereinafter, sometimes referred to as “the ECL detection method”) uses the above-described ECL sensor. The ECL detection method of the present invention may include: an enzyme reaction step of adding a sample to be measured to a Cas enzyme-catalyzed system including a guide nucleic acid capable of binding to a target nucleic acid to obtain a sample reaction solution; and a sample detection step of adding the sample reaction solution to the electrochemiluminescent sensor and collecting and analyzing an electrochemiluminescent signal.

In the ECL detection method of the present invention, the ECL sensor of the present invention can be produced at the time of use, instead of using the ECL sensor as it is. In this case, the ECL detection method of the present invention may further include a hot exciton chip preparation step of dropping the ECL nanoprobe of the present invention onto a working electrode and thereby obtaining a hot exciton chip of the present invention (that is, the ECL sensor).

With the ECL detection method of the present invention, a target molecule contained in the sample to be measured can be detected. The sample to be measured is not limited to a particular one, and may be, for example, urine, blood, serum, cerebrospinal fluid, or saliva. The target molecule is not limited to a particular one, and may be, for example, a nucleic acid, protein, or a chemical small molecule.

In the present invention, when an enzyme-catalyzed reaction or another specific chemical reaction is used as a target-identification and signal-conversion unit and the target molecule is dropped, a signal switch state of the ECL nanoprobe can be changed through the enzyme-catalyzed reaction or the chemical reaction, and thus qualitative or quantitative detection for a target signal can be realized.

As an enzyme used for the enzyme-catalyzed reaction, for example, a Cas enzyme having a collateral cleavage activity in a CRISPR/Cas enzyme can be used. The above-mentioned activity belongs to a nonspecific nuclease cleavage activity. When the Cas enzyme is targeted as a target nucleic acid molecule, the Cas enzyme is activated and acquires the nonspecific activity for the cleavage of any nucleic acid molecule.

The Cas enzyme may include at least one Cas protein selected from the group consisting of Cas12 (type VA), Cas13 (type VI), and Cas14 (type VF), and is, for example, at least one Cas protein selected from the group consisting of Cas12a, Cas12b, Cas13a, Cas13b, Cas14a, Cas14b, and Cas14c. Here, Cas12 targets dsDNA (double-stranded DNA), Cas13 targets ssRNA (single-stranded RNA), and Cas14 targets ssDNA. Furthermore, Cas12a has a nonspecific cleavage activity (also called a trans-cleavage activity) for ssDNA (single-stranded DNA), and is therefore capable of targeting ssDNA.

The Cas12 enzyme targets DNA, is commercially mature, and can be used for other detection areas for non-nucleic acids (for example, protein and a small biomolecule) in addition to DNA, and is therefore more preferably used.

In the present invention, any nucleic acid sequence can be targeted by designing and synthesizing a guide RNA (gRNA) for a Cas enzyme. The gRNA is composed of two parts: one is CRISPR RNA (crRNA), that is, a 17 to 20-base length nucleotide sequence complementary paired with the target nucleic acid; and the other one is tracrRNA serving as an anchor to aid in the folding of the Cas enzyme. The guide RNA activates the Cas enzyme by binding to oligonucleotide in the target nucleic acid. The activated Cas enzyme causes the cleavage of the oligonucleotide bound to the ECL nanoprobe to occur by the collateral cleavage activity and thereby changes a signal switch state of the ECL nanoprobe.

Hereinafter, the principle of a signal switch and a method for the production of and the detection by an ECL detection chip according to the present invention will be specifically described using, as examples, hot exciton organic nanorods (BB NRs) and a sensor array produced in the examples of the present invention.

The principle of the signal switch of the present invention will be specifically described using the ECL detection chip illustrated in FIG. 2 as an example. FIG. 2 is a schematic diagram illustrating an ECL switch based on an ssDNA-BB NRs nanoprobe according to the present invention. By the binding of the hot exciton nanoparticles (BB NRs) to oligonucleotide (ssDNA) modified with a quencher molecule (BHQ) at an end thereof, an ECL nanoprobe (the ssDNA-BB NRs nanoprobe) of the present invention is obtained and used as a reporter probe in ECL detection. The end of ssDNA is modified with the BHQ, hence the BB NRs are in the ECL OFF state. The ssDNA-BB NRs nanoprobe is dropped directly onto a surface of an ITO electrode to construct an ECL sensor interface. In the case where a Cas enzyme (Cas12a enzyme) is not activated when the reaction solution is dropped onto the sensor interface, the BHQ is bound to surfaces of the BB NRs via the ssDNA and the sensor electrode indicates a low ECL intensity (ECL OFF). On the contrary, in the case where a target molecule is present and the Cas12a enzyme is activated, the cleavage of the ssDNA occurs, whereby the BHQ is separated from the surfaces of the BB NRs, ECL of the BB Nrs is recovered, and a strong ECL signal is generated in the sensor interface (ECL ON).

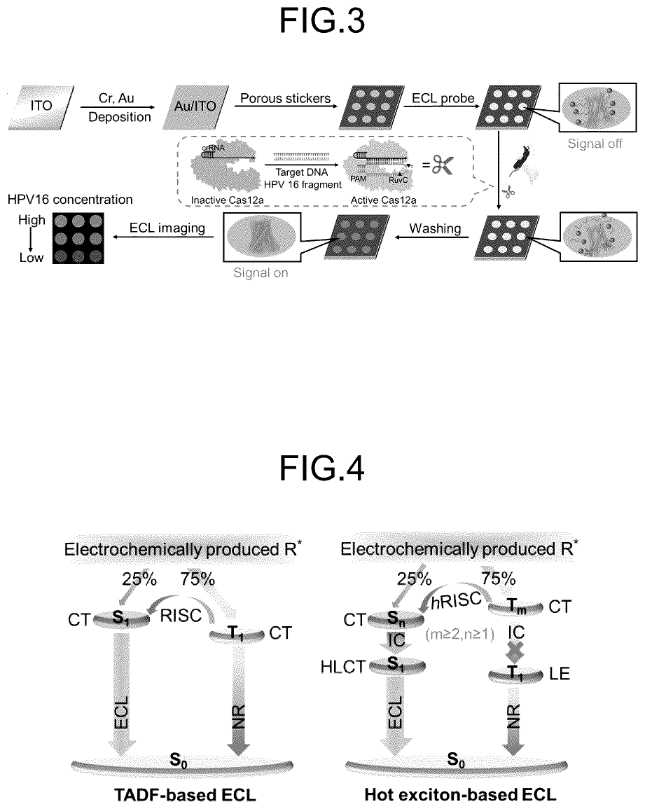

The method for the production of and detection by the ECL detection chip will be specifically described using an ECL detection method illustrated in FIG. 3 as an example. FIG. 3 is a schematic diagram illustrating the production of and detection by a sensor array based on ssDNA-BB NRs according to the present invention. First, the ECL nanoprobe of the present invention is dropped onto an indium tin oxide (ITO) electrode or a gold-indium tin oxide (Au/ITO) electrode obtained by deposition of gold and other metals, whereby an ECL electrode (that is, the ECL sensor) of the present invention is obtained. A reaction solution containing Cas protein, crRNA and a possibly-contained target nucleic acid to be measured is dropped onto the ECL electrode, and, when the target molecule is absent and the Cas enzyme is not activated owing to the above-described signal switch, the sensor electrode indicates a low ECL intensity (ECL OFF). On the contrary, when the target molecule is present and the Cas12a enzyme is activated, the ECL of the BB NRs is recovered and a strong ECL signal (ECL ON) is generated in the sensor interfaces, whereby electrochemiluminescence detection based on the hot exciton organic luminescent molecule of the present invention can be achieved.

From the viewpoint of achieving higher ECL signal intensity, ECL detection conditions, such as the concentration of the Cas protein in a sample reaction solution, the usage ratio of the Cas protein to the crRNA, and a reaction time (for example, an incubation time) of the sample reaction solution on the chip, are preferably optimized.

The concentration of the Cas protein is not particularly limited, but may be, for example, 40 nM or higher, preferably 42 nM or higher, more preferably within a range of 45 nM to 70 nM, and particularly preferably within a range of 50 nM to 60 nM from the viewpoint of achieving higher ECL signal intensity.

Although the usage ratio of the Cas protein to the crRNA is not particularly limited, but, from the viewpoint of achieving higher ECL signal intensity, the amount of the crRNA used is preferably larger than the amount of the Cas protein used. For example, the usage ratio of the Cas protein to the crRNA may be within a range of 1:1.25 to 1:5, preferably within a range of 1:1.35 to 1:4, more preferably within a range of 1:1.5 to 1:3, and particularly preferably within a range of 1:1.5 to 1:2.

The incubation time of the sample reaction solution on the chip is not particularly limited, but may be, for example, 30 minutes or longer, preferably within a range of 35 to 120 minutes, more preferably within a range of 37 to 90 minutes or longer, and particularly preferably within a range of 40 to 60 minutes from the viewpoint of achieving higher ECL signal intensity.

As verified by the later-described examples of the present invention, the ECL efficiency of the hot exciton material of the present invention can reach 56.7% and the fluorescence quantum yield (Φ_{PL}) can reach 89% or higher. The hot exciton material of the present invention is higher in ECL efficiency and fluorescence quantum yield than the previously-reported Pdots based on the principle of the thermally activated delayed fluorescence (TADF) (that have the reported highest organic ECL nanomaterial efficiency) (Anal. Chem. 2022, 94, 15695-15702) and Pdots based on a conventional fluorescent material (Chem. Sci. 2019, 10, 6815; Anal. Chem. 2016, 88, 845).

FIG. 4 is a schematic diagram contrastively illustrating the luminescence principle of a TADF compound and the luminescence principle of the hot exciton material. As illustrated in FIG. 4, the RISC process for the TADF compound occurs between T₁ and S₁, and therefore the TADF compound is called a cold exciton material. The charge transfer (CT) properties of both the lowest singlet (S₁) and triplet (T₁) excited states of the TADF compound are given lower Φ_{PL}, and also moderate reverse intersystem crossing (RISC) from T₁ to S₁ of the TADF compound easily causes the accumulation of long-lived triplet excitons, which results in serious extinction. Contrary to a cold exciton path of the TADF compound, the RISC of the hot exciton material occurs in a high energy level excited state (hRISC, Tₘ→Sₙ, m≥2, n≥1). Here, a large energy gap Tₘ-T₁ suppresses internal conversion (IC) from Tₘ to T₁, meanwhile narrow energy splitting Tₘ-Sₙ accelerated hRISC. Thus, owing to the accelerated RISC, the hot exciton material more sufficiently utilizes a triplet exciton than the TADF material. The hRISC process reduces T₁ exciton quenching. Also, unlike the strong CT properties of the TADF material, the hot exciton material has HLCT excited state properties. Here, the S₁ state is an LE state or an HLCT state dominated by LE, and the Tₘ state and the Sₙ state are CT states, and therefore high φ_{PL} and 100% exciton utilization efficiency are given.

Using the detection of HPV16 DNA as an example, the ECL detection method according to the present invention will be compared with other methods described in the prior art, in terms of detection range and detection limit. Comparison results are illustrated in Table 1.

TABLE 1

| | Detection Range | Detection |
|---|---|---|
| Method | (nM) | Result |
| Electrochemiluminescence¹ | 1 TO 100 | 0.6 nM |
| Electrochemiluminescence² | 0.1 TO 200 | 0.03 nM |
| Electrochemiluminescence³ | 0.001 TO 10 | 0.48 pM |
| Electrochemiluminescence⁴ | 0.00001 TO 0.015 | 7.6 fM |
| Electrochemical⁵ | 0.5 TO 100 | 150 pM |
| Electrochemical⁶ | 0.0000001-0.01 | 18.6 aM |
| Present Invention | 0.000001-10 | 0.6 fM |

Note that superior FIGS. 1 to 6 indicate the following references, respectively.
¹L. Wang et al., Microchem. J. 2022, 181, 107818.
²Y. Nie et al., Biosens. Bioelcetron. 2020, 160, 112217.
³P. Liu et al., Biosens. Bioelectron. 2021, 176, 112954.
⁴Y. He et al., ACS Appl. Mater. Interfaces, 2021, 13, 298.
⁵S. Jampasa et al., Sensor. Actuat. B-Chem, 2018, 265, 514.
⁶Y. He et al., Sensor. Actuat. B-Chem, 2020, 320, 128407.

Note that superior FIGS. 1 to 6 indicate the following references, respectively.
1: L. Wang et al., Microchem. J. 2022, 181, 107818.
2: Y. Nie et al., Biosens. Bioelectron. 2020, 160, 112217.
3: P. Liu et al., Biosens. Bioelectron. 2021, 176, 112954.
4: Y. He et al., ACS Appl. Mater. Interfaces, 2021, 13, 298.
5: S. Jampasa et al., Sensor. Actuat. B-Chem, 2018, 265, 514.
6: Y. He et al., Sensor. Actuat. B-Chem, 2020, 320, 128407.

As seen from Table 1, the ECL chip (sensor) produced based on the hot exciton organic luminescent molecule of the present invention has a wider detection range and higher sensitivity than the other reported ECL chips.

### ECL Detection Kit

An ECL detection kit of the present invention may directly include the above-described ECL sensor of the present invention. The ECL detection kit beneficially includes the above-described ECL nanoprobe of the present invention and drops the ECL nanoprobe onto a working electrode during detection. Besides the ECL sensor, the ECL detection kit may include, for example, other reagents necessary for ECL detection, such as a Cas enzyme-containing detection reagent, and an instruction manual.

The electrochemiluminescent nanoprobe, the method for producing the electrochemiluminescent nanoprobe, and the application of the electrochemiluminescent nanoprobe, and the like according to the present invention have been described above, based on the embodiments, but the present invention is not limited to them. Other aspects obtained by applying various modifications conceivable by those skilled in the art to the embodiments and other aspects constructed by combining some of the constituents in the embodiments are also included in the scope of the present invention as long as the aspects do not deviate from the gist of the present invention.

### EXAMPLES

Hereinafter, the hot exciton nanoparticles, the ECL nanoprobe, the ECL detection method, and the like according to the present invention will be specifically described with reference to examples. Note that the present invention is not limited to these examples.

Reagents, an experimental method, and apparatuses that are used in the examples are as follows.

### (1) Reagents

Tetrakis(triphenylphosphine)palladium (Pd(Pph₃)₄), purchased from Bide Pharmaceutical Technology Co., Ltd. (Shanghai, China).
4-(9H-carbazol-9-yl)phenylboronic acid pinacol ester, purchased from Macklin Biochemical Technology Co., Ltd. (Shanghai, China).
4,7-dibromobenzo[c]-1,2,5-thiadiazole, purchased from Aladdin Biotech Co., Ltd. (Shanghai, China).
EnGen (registered trademark) Lba Cas12a (10-μM solution), purchased from New England Biolabs (Ipswich, MA, UK).
Polystyrene-maleic anhydride (PSMA, average Mw: 1700), 4-(2-hydroxyethyl)-1-piperazine ethanesulfonic acid (HEPES), polyethylene glycol (PEG, average Mw: 3350), 1-(3-(dimethylamino)propyl)-3-ethylcarbodiimide hydrochloride (EDC), and tetrabutylammonium chloride, all purchased from Sigma-Aldrich Co., Ltd. (Shanghai, China).
Tripropylamine (TPrA, >98%), purchased from J&K Chemical Ltd. (Beijing, China).
An indium tin oxide (ITO) glass sheet (100×100 mm×1.1 mm), purchased from Zhuhai Kaivo Optoelectronic Technology Co., Ltd. (Zhuhai, China).
Diethyl carbonate (DEPC) water and all oligonucleotides listed in Table 2, all purchased from Sangon Bioengineering Co. Ltd. (Shanghai, China).
A phosphate buffer solution (PBS, 0.1 M, pH 7.4) prepared by mixing a KH₂PO₄ storage solution with a Na₂HPO₄ storage solution.

Oligonucleotide sequences used in the examples are as follows.

TABLE 2

| Sequence | | |
|---|---|---|
| Number | Name | Sequence (5′-3′) |
| 1 | HPV 16-TS | AAT ATG TCA TTA TGT GCT GCC ATA TCT |
| | | ACT TCA GAA ACT |
| 2 | HPV 16-NTS | AGT TTC TGAAGT AGA TAT GGC AGC ACA |
| | | TAA TGA CAT ATT |
| 3 | HPV 18-TS | AAT TTAACAATA TGT GCT TCT ACA CAG |
| | | TCT CCT GTA CCT |
| 4 | HPV 18-NTS | AGG TAC AGG AGA CTG TGT AGA AGC ACA |
| | | TAT TGT TAA ATT |
| 5 | HPV 31-TS | GGT GAA CCG AAAACG GTT GGT ATA TAA |
| | | AGC ACA TAG TAT |
| 6 | HPV 31-NTS | ATA CTA TGT GCT TTA TAT ACC AAC CGT |
| | | TTT CGG TTC ACC |
| 7 | HPV 58-TS | ACA GCT AGG GCA CAC AAT GGT ACA TGT |
| | | GCC CAT AAG CAG |
| 8 | HPV 58-NTS | CTG CTT ATG GGC ACA TGT ACC ATT GTG |
| | | TGC CCT AGC TGT |
| 9 | SSDNA-BHQ2 | NH₂-C₆-TT TTT ATT TTT-BHQ2 |
| 10 | crRNA | UAA UUU CUA CUA AGU GUA GAU UGAAGU |
| | (HPV16) | AGA UAU GGC AGC AC |

### (2) Experimental Method and Apparatus

TEM is obtained by TECNAIG2F 20 transmission electron microscope (FFI, USA).

Zeta potentials are recorded using 90 Plus DynaPro NanoStar (Brookhaven Instrument Corporation, USA).

¹H NM/R spectra are measured using deuterated CDCl₃ as a solvent and tetramethylsilane (TMS) as an internal standard by Bruker Avance III 500HD spectrometer (Germany).

UV-vis absorption spectra are obtained by UV-3600 spectrophotometer (Shimadzu, Japan).

Photoluminescence (PL) spectra and fluorescence lifetimes are measured using FLS-980 fluorescence spectrophotometer (Edinburgh, U.K).

AFM is obtained in the ScanAsyst mode by an atomic force microscope (BRUKER Dimension Icon, Germany).

A 5-nm Cr layer and a 50-nm Au layer are deposited on an ITO glass sheet by PVD 75 electron beam deposition system (Kurt J. Lesker, USA).

ECL spectra are obtained by an ECL spectrometer produced by Prof. Guizheng Zhou's laboratory at Shandong University. The ECL spectrometer is composed of Acton SP 2300i monochromator including PyLoN 400BR-eXcelon digital CCD detector (Princeton, USA) to be cooled by liquid N₂ and VersaSTAT3 electrochemical analyzer (Princeton, USA).

Cyclic voltammetry experiments are performed on CHI 630D electrochemical workstation (CHI instruments Inc., China).

Anodic and cathodic ECL experiments are performed in an in-house manufactured reaction vessel of an MPI-EII ECL analyzer (Xi'an Remex, China). The analyzer employs a three-electrode system.

ECL imaging is performed with an in-house manufactured ECL imaging system. The imaging system includes a focusing lens (EF 50 mm f/1.2 L USM, Canon), an electron multiplying charge coupled device (EMCCD, iXon Ultra, Andor, UK), and a CHI-660D electrochemical workstation. An Au/ITO electrode is used as a working electrode, Ag/AgCl wire is used as a reference electrode, and Pt wire is used as a counter electrode.

### Example 1: Production and Characteristics Analysis of Hot Exciton Organic Nanorods (BB NRs)

a. Synthesis of Hot Exciton Molecule BCzP-BT

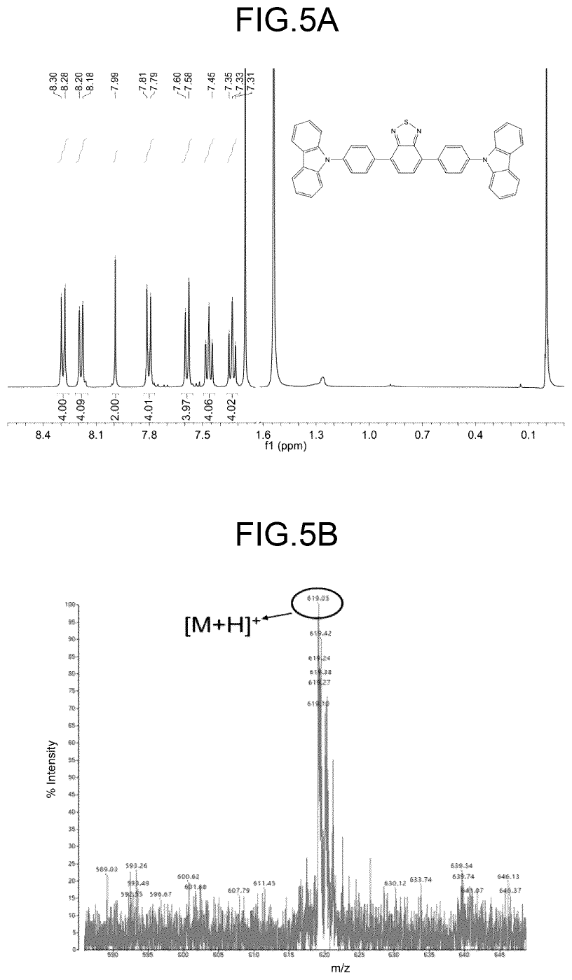

First, 4-(9H-carbazol-9-yl)phenylboronic acid pinacol ester (Cz, 1.1817 g, 3.2 mmol), 4,7-dibromobenzo[c]-1,2,5-thiadiazole (BT, 0.4145 g, 1.41 mmol), K₂CO₃ (2.0040 g, 14.5 mmol), and tetrakis(triphenylphosphine)palladium (Pd(Pph₃)₄, 0.2311 g, 0.2 mmol) were added to a mixed solution of 20 mL of toluene and 4 mL of water contained in a Schlenk tube, and then the resulting mixed solution was stirred in the Schlenk tube at 90° C. for 18 hours in an atmosphere of argon. Subsequently, the reacted mixed solution was extracted with dichloromethane by using petroleum ether and dichloromethane as eluents and purified by silica gel column chromatography, whereby 0.7512 g of a BCzP-BT product (that is, 4,7-bis(4-(9H-carbazol-9-yl)phenyl)benzo[c][1,2,5]thiadiazole (p-BCzP-BT), hereinafter abbreviated as “BCzP-BT”) was obtained and the yield thereof was 86%. The synthesis of the BCzP-BT molecule was verified by nuclear magnetism (¹H NMR) and a mass spectrum. The results are illustrated in FIG. 5A and FIG. 5B. FIG. 5A and FIG. 5B are diagrams illustrating the results of nuclear magnetic (¹H NMR) and mass spectrometric analysis of the BCzP-BT molecule produced in Example 1 of the present invention. Here, FIG. 5A is a diagram illustrating the result of the nuclear magnetic (¹H NMR) analysis of the BCzP-BT molecule, and FIG. 5B is a diagram illustrating the result of the mass spectrometric analysis of the BCzP-BT molecule.

FIG. 5A is a diagram illustrating the result of the nuclear magnetic (¹H NMR) analysis of the BCzP-BT molecule produced in Example 1 of the present invention, and FIG. 5B is a diagram illustrating the result of the mass spectrometric analysis of the BCzP-BT molecule. As seen from FIG. 5A and FIG. 5B, the present invention successfully synthesized the BCzP-BT molecule.

b. Production of Hot Exciton Organic Nanorods (BB NRs)

First, 2.5 mL of a THF solution containing 50 μg/mL of BCzP-BT and 10 μg/mL of a polystyrene-maleic anhydride copolymer (PSMA, Mn=1700) was ultrasonically treated at room temperature for 20 minutes. The solution was then quickly added to 10 mL of water and ultrasonically treated for 30 seconds. By rotary evaporation at 35° C., the solvent THF was removed, and the resultant solution was further filtered through a polyethersulfone syringe filter to obtain BB NRs.

c. Excited State and Characteristics Analysis of BCzP-BT Molecule

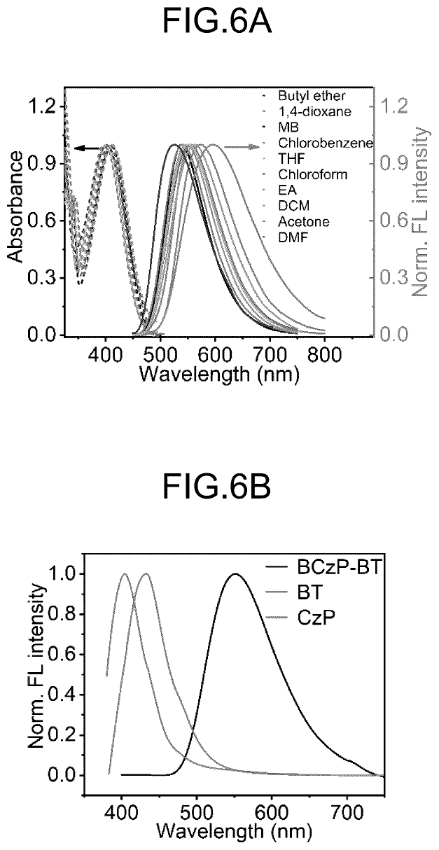

An excited state of the BCzP-BT molecule produced in Example 1 was analyzed. The results of the analysis are illustrated in FIG. 6A and FIG. 6B. FIG. 6A and FIG. 6B are diagrams illustrating the analysis results of the excited state of the BCzP-BT molecule produced in Example 1 of the present invention. Here, FIG. 6A is a diagram illustrating normalized ultraviolet-visible absorbance and FL spectra of BCzP-BT in different polar solvents, and FIG. 6B is a diagram illustrating FL spectra of CzP and BT in the BCzP-BT molecule and BCzP-BT in a 10⁻⁵M THF solution.

First, normalized ultraviolet-visible absorbance and fluorescence (FL) spectra of the BCzP-BT molecule in different polar solvents were examined. The results are illustrated in FIG. 6A. Owing to the transfer of intramolecular charge from Cz to BT unit, BCzP-BT exhibited a broad absorbance at approximately 400 nm, and the shape and position of the ultraviolet absorption peak did not greatly change with an increase in solvent polarity. However, with the increase in the solvent polarity, the fluorescence spectrum of BCzP-BT exhibited a red shift of 68 nm and a broader fluorescence peak, which indicated that the BCzP-BT molecule has a solvent effect. As seen from FIG. 6A, the BCzP-BT molecule has strong charge transfer (CT) state characteristics.

Subsequently, differences in FL spectra of CzP and BT in the BCzP-BT molecule and BCzP-BT in the 10⁻⁵M THF solution were analyzed. The results are illustrated in FIG. 6B. As seen from FIG. 6B, the luminescence of the BCzP-BT molecule is caused by the charge transfer from Cz to BT and is not originated from the monomer itself.

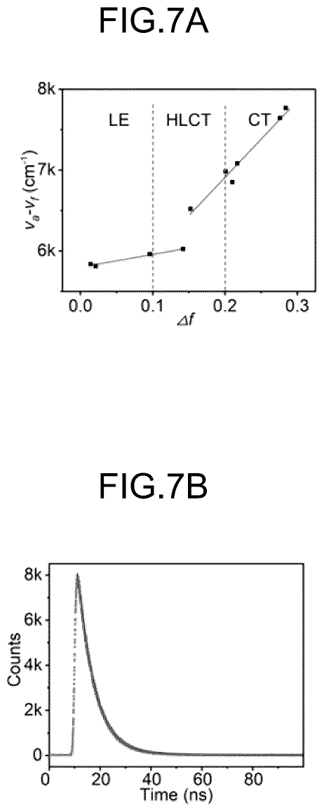

FIG. 7A and FIG. 7B are diagrams illustrating properties of the excited states of the BCzP-BT molecule produced in Example 1 of the present invention. Here, FIG. 7A is a diagram illustrating a variation curve of Stokes displacement (vₐ-v_{f}) and the orientation polarizability (Δf) of the solvent, and FIG. 7B is a diagram illustrating a transient PL decay curve of BCzP-BT in THF.

Furthermore, to study the excited state properties of BCzP-BT, a variation curve of Stokes displacement (vₐ-v_{f}) and the orientation polarizability (Δf) of the solvent was drawn, based on a Lippert-Mataga solvent-induced color change model, and a dipole moment of S₁ was calculated based on the slope of the curve. The results are illustrated in FIG. 7A.

In FIG. 7A, two cross-sectional fitting lines are observed. A small dipole moment of a low-polarity solvent and a larger dipole moment of a high-polarity solvent correspond to a locally excited (LE) state and a charge transfer (CT) excited state, respectively. For a medium-polarity solvent, the excited state exhibited the LE state and the CT excited state at the same time.

Furthermore, the fluorescence lifetime of BCzP-BT was analyzed. The results are illustrated in FIG. 7B. FIG. 7B is a diagram illustrating the transient PL decay curve of BCzP-BT in THF. As seen from FIG. 7B, BCzP-BT exhibited a single-index decay of 6.72 ns with no delay. Furthermore, the results proved that the S₁ excited state of BCzP-BT was a hybrid locally-excited and charge-transfer excited state (HLCT) and was not a simple mixture of the LE and CT states.

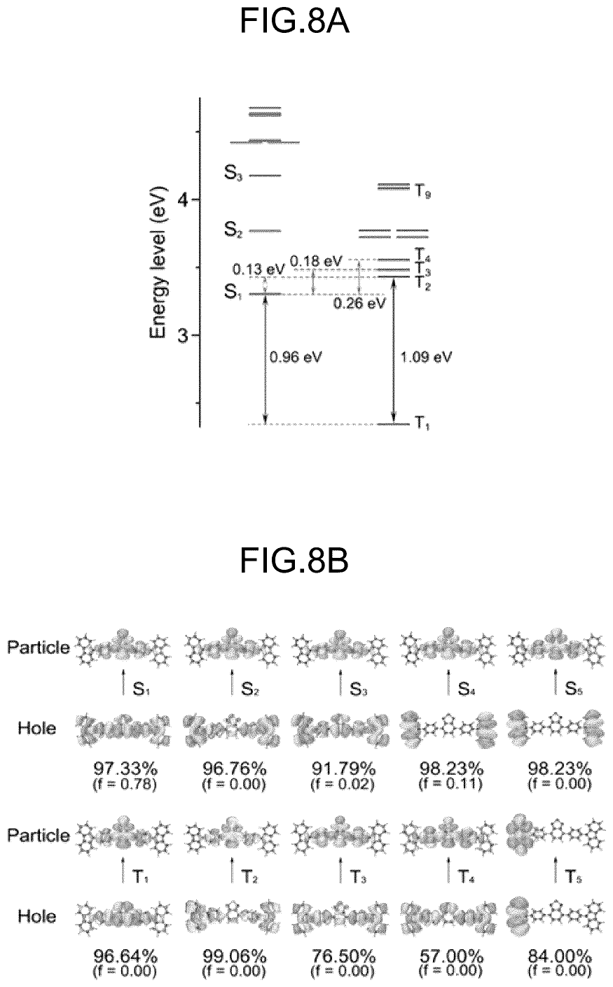

FIG. 8A and FIG. 8B are diagrams illustrating analysis results of possibilities of a hot exciton process for the BCzP-BT produced in Example 1 of the present invention. Here, FIG. 8A is a diagram illustrating the energy level distribution of the BCzP-BT molecule, and FIG. 8B is a diagram illustrating the results of natural transition orbital simulation of the BCzP-BT molecule.

To verify the possibility of the hot exciton process, the energy levels of the lowest singlet and triplet excited states of BCzP-BT were calculated using TD-m062x/6-311g(d). The results are illustrated in FIG. 8A.

The results illustrated in FIG. 8A reveals that a band gap between T₂ and T₁ (1.09 eV) suppresses triplet internal conversion and a narrow gap between T₂ and S₁ (0.13 eV) accelerates reverse intersystem crossing, whereby quenching caused by triplet exciton accumulation can be reduced and triplet excitons can be more effectively used via a hot exciton channel.

Furthermore, natural transition orbital (NTO) simulations were performed for the BCzP-BT molecule by using the Multiwfn software. The simulation results are illustrated in FIG. 8B. The results illustrated in FIG. 8B reveals that T₁ is characterized by having a locally-excited (LE) state, Sₘ and Tₙ are characterized by having charge-transfer (CT) excited states, and S₁ is characterized by having a hybrid locally-excited and charge-transfer excited state (because electrons in the S₁ state are concentrated on BT and a benzene ring adjacent thereto and holes are distributed throughout a skeleton).

d. Characteristics Analysis of BB NRs

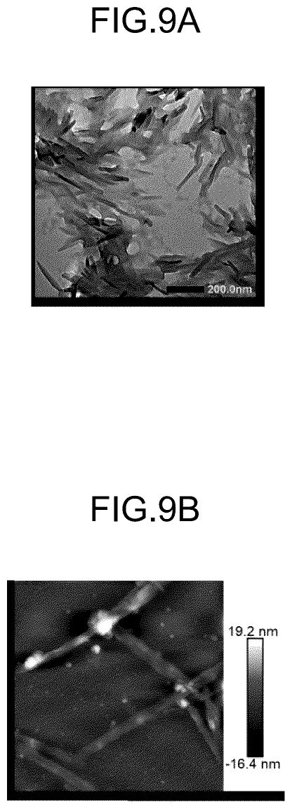

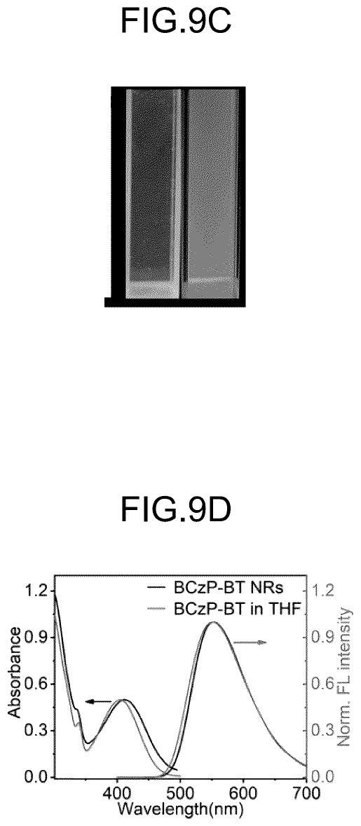

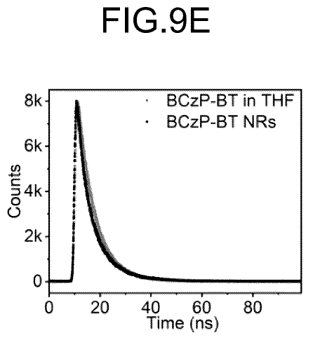

The characteristics of the BB NRs produced in Example 1 of the present invention were analyzed. The results are illustrated in FIG. 9A to FIG. 9E. FIG. 9A to FIG. 9E are diagrams illustrating the analysis results of characteristics of the BB NRs produced in Example 1 of the present invention. Here, FIG. 9A is a TEM image, FIG. 9B is an AFM image, and FIG. 9C, FIG. 9D, and FIG. 9E are diagrams illustrating the ultraviolet spectra, the fluorescence spectra, and the fluorescence lifetimes of BB NRs and BCzP-BT, respectively.

First, the form of synthesized BB NRs was analyzed by TEM and AFM. The TEM and AFM images are illustrated in FIG. 9A and FIG. 9B, respectively. The results illustrated in FIG. 9A and FIG. 9B reveals that the BB NRs are in the form of rods each having a width of approximately 30 nm and a length of approximately 200 nm.

FIG. 9C, FIG. 9D, and FIG. 9E are diagrams illustrating the ultraviolet spectra, the fluorescence spectra, and the fluorescence lifetimes of the BB NRs and the BCzP-BT, respectively. As seen from FIG. 9C, a BB NRs solution is light green in sunlight and emits a bright yellow-green light under 365 nm ultraviolet irradiation. As seen from FIG. 9D, compared to the BCzP-BT, the BB NRs have the possibility of lowering the transition energy in a polar environment in water, and hence the ultraviolet absorption of the BB NRs exhibits a slight red-shift at approximately 400 nm. As seen from FIG. 9D, both the BCzP-BT and the BB-NRs exhibited similar FL emission at 554 nm. As seen from FIG. 9E, both the BCzP-BT and the BB-NRs have a FL lifetime of approximately 6.7 ns. These results revealed that BB-NR has the same photophysical properties as those of the BCzP-BT.

e. ECL Characteristics of BB NRs

The ECL characteristics of the BB NRs produced in Example 1 of the present invention were analyzed.

First, the stability of a radical intermediate of the BB NRs was examined by conducting an annihilation ECL experiment. Note that specific conditions of the annihilation ECL experiment are as follows.
Electrode material: BB NRs/GCE
Electrolyte: 0.1M PBS pH 7.4, nitrogen gas ventilation for 20 minutes
ECL: cyclic voltammetry
Annihilated state ECL: pulse step method
Maximum potential: +1.5 V
Minimum potential: −2.0 V
Scanning speed: 0.1 V/s
PMT: High voltage of 800 V

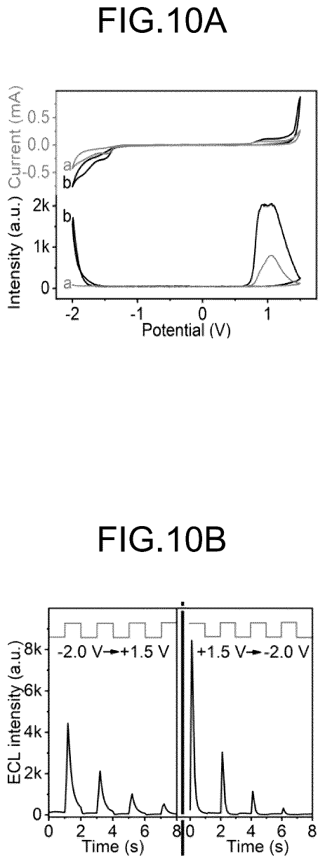

The results of the experiment are illustrated in FIG. 10A and FIG. 10B. FIG. 10A and FIG. 10B are diagrams illustrating analysis results of ECL characteristics of the BB NRs produced in Example 1 of the present invention. Here, FIG. 10A is a diagram illustrating cyclic voltammetry (CV) curves and ECL curves of BB NRs/GCE. Curves a indicate the results of anodic scanning performed first. Curves b indicate the results of cathodic scanning performed first. FIG. 10B is a diagram illustrating annihilated state ECL curves of the BB NRs/GCE.

FIG. 10A is a diagram illustrating the cyclic voltammetry (CV) curves and the ECL curves of the BB NRs/GCE. As illustrated in FIG. 10A, in PBS saturated with N₂, the cyclic voltammetry (CV) curve of a glass carbon electrode modified with the BB NRs (BB NRs/GCE) exhibited an oxidation peak at +0.95 V (corresponding to the oxidation of Cz) and exhibited a reduction peak at −1.45 V (corresponding to the reduction of BT).

In addition, as seen from FIG. 10A, a scanning direction has a great effect on ECL. When anodic scanning was performed first (the curves a), weak ECL emission (from 0 to +1.5 V and further to −2.0 V) appeared only at +1.05 V. When cathodic scanning was performed first (the curves b), strong ECL emission (from 0 to −2.0 V and further to +1.5 V) appeared at both +1.0 V and −2.0 V. These results proved that the reduced state of the BB NRs was more stable.

At the same time, the annihilated state ECL of the BB NRs was detected using the pulse step method. The results are illustrated in FIG. 10B. In FIG. 10B, the left-side part indicates −2.0 to +1.5 V and the right-side part indicates +1.5 to −2.0 V. According to the results in FIG. 10B, regardless of whether an initial potential is negative or positive, transient ECL emission of the BB NRs was observed only at +1.50 V, which proved that the reduced state of the BB NRs had higher stability.

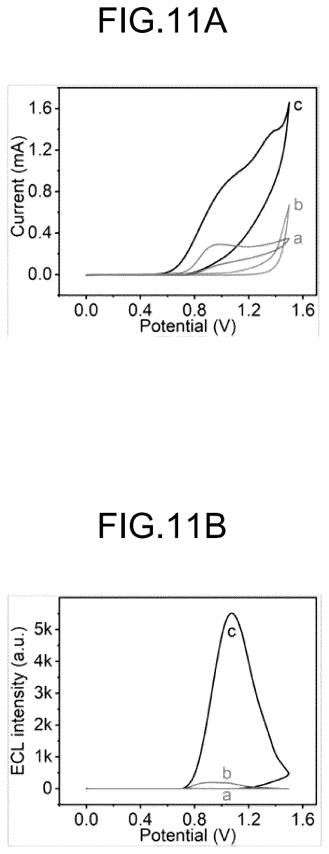

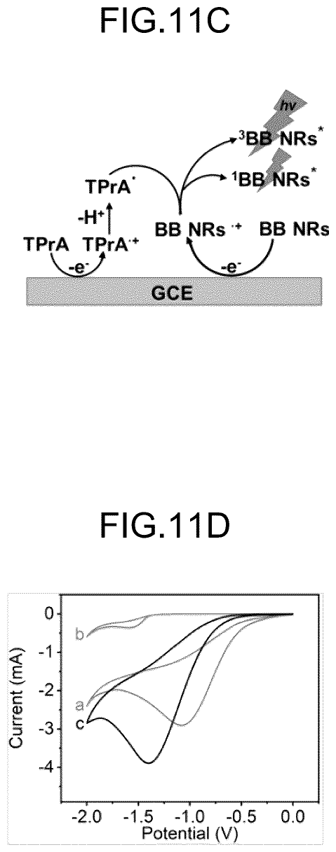

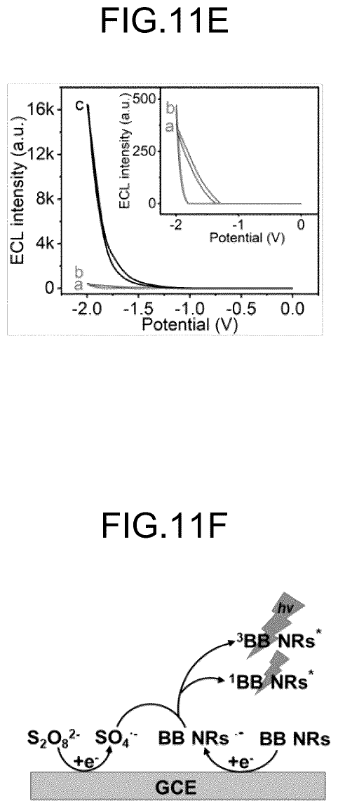

The ECL of the BB NRs in the presence of a coreactant was further analyzed. The results of the analysis are illustrated in FIG. 11.

First, an anodic ECL behavior of BB NRs was examined using tripropylamine (TPrA) as a coreactant.

Specific experimental conditions for the anodic ECL experiment are as follows.
Electrode material: BB NRs/GCE
Electrolyte: 10-mM TPrA-containing 0.1M PBS, pH 7.4
Test method: cyclic voltammetry
Maximum potential: +1.5 V
Minimum potential: 0 V
Scanning speed: 0.1 V/s
ECL: PMT, high voltage of 400 V
Furthermore, a cathodic ECL behavior of the BB NRs was examined using S₂O₈²⁻ as a co-reactor.
Specific experimental conditions for the cathode ECL experiment are as follows.
Electrode material: BB NRs/GCE
Electrolyte: 100-mM K₂S₂O₈-containing 0.1 M PBS, pH 7.4
Test method: cyclic voltammetry
Maximum potential: 0 V
Minimum potential: −2.0 V
Scanning speed: 0.1 V/s
ECL: PMT, high voltage of 400 V

The results of the analysis are illustrated in FIG. 11A to FIG. 11F. FIG. 11A to FIG. 11F are diagrams illustrating the analysis results of ECL of the BB NRs produced in Example 1 of the present invention, in the presence of a co-reactive agent. Here, in FIG. 11A, FIG. 11B, and FIG. 11C, anodic ECL behaviors and mechanisms of the BB NRs when tripropylamine (TPrA) is used as a coreactant are illustrated. In FIG. 11A and FIG. 11B, curves a indicate TPrA itself, curves b indicate the absence of TPrA, and curves c indicate coexistence with TPrA. In FIG. 11D, FIG. 11E, and FIG. 11F, cathodic ECL behaviors and mechanisms of BB NRs when K₂S₂O₈ is used as a coreactant are illustrated. In FIG. 11D and FIG. 11E, curves a indicate S₂O₈⁻² itself, curves b indicate the absence of S₂O₈²⁻, and curves c indicate coexistence with S₂O₈²⁻.

As seen from FIG. 11A, an irreversible oxidation peak of tripropylamine (TPrA) itself (the curves a) is observed at +0.98 V, and a starting potential thereof is +0.57 V. Note that a TPrA· radical is formed after deprotonation of TPrA·⁺ (a TPrA oxidation product). As seen from FIG. 11A and FIG. 11B, in the case of the absence of TPrA (the curves b), oxidation of the BB NRs started at approximately +0.70 V, and cation radical BB NRs·⁺ were formed. This led to a clear oxidation current and weak ECL emission. As seen from FIG. 11A and FIG. 11B, in the case of coexistence with TPrA (the curves c), the BB NRs/GCE exhibited oxidation peaks of TPrA and BB NRs and strong ECL emission having a peak at +1.07 V. It can be considered that this phenomenon is caused by radiative relaxation of singlet and triplet state BB NRs (¹BB NRs* and ³BB NRs*) excited by charge transfer between TPrA· and BB NR·⁺ (see FIG. 11C).

As seen from FIG. 11D and FIG. 11E, S₂O₈²⁻ itself (curves a) can be reduced to strong oxidizing SO₄·⁻ at −1.07 V, but ECL emission thereof is low and negligible. As seen from FIG. 11D and FIG. 11E, in the case of the absence of K₂S₂O₈ (curves b), the BB NRs/GCE had a clear reduction peak at −1.55 V and the BB NRs can be reduced to BB NRs·⁻ with weak ECL emission. As seen from FIG. 11D and FIG. 11E, in the case of the presence of K₂S₂O₈ (curves c), the BB NRs/GCEs generated a clear reduction current and had a peak potential of −1.40 V and also involved strong ECL emission. It can be considered that this phenomenon is caused by generating ¹BB NRs* and ³BB NRs* emission owing to a reaction of BB NRs·⁻ with SO₄⁻ (see FIG. 11F).

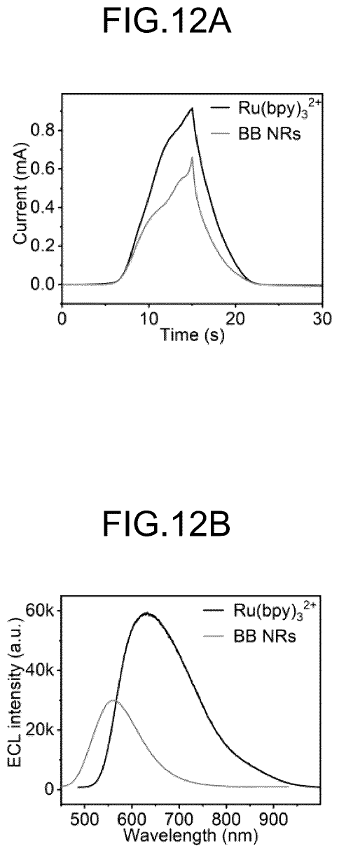

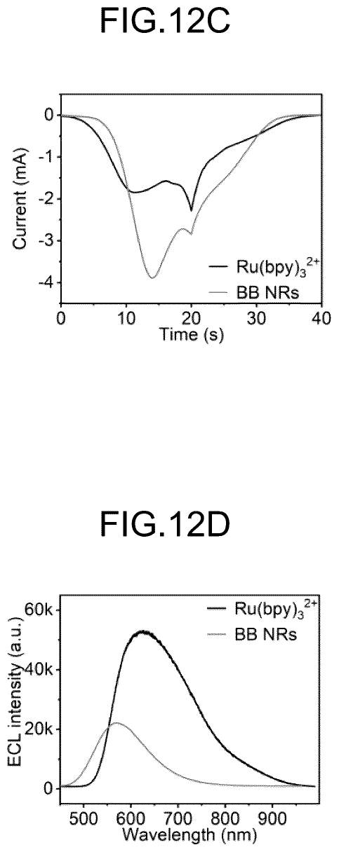

Furthermore, the ECL efficiency of BB NRs was verified by recording a current-time curve and ECL spectra of BB NRs by using 1 mM of Ru(bpy)₃²⁺ as a standard in the presence of 10 mM of TPrA· or 100 mM of K₂S₂O₈. Note that specific experimental conditions are the same as for the anodic and cathodic ECL experiments described above. The results are illustrated in FIG. 12A to FIG. 12D and Table 3. FIG. 12A to FIG. 12D are diagrams illustrating the ECL efficiency of BB NRs produced in Example 1 of the present invention, based on Ru (bpy)₃²⁺. Here, FIG. 12A and FIG. 12B are diagrams illustrating anodic current-time curves and ECL spectra of BB NRs-modified and Ru(bpy)₃²⁺-modified GCE, respectively. FIG. 12C and FIG. 12D are diagrams illustrating cathodic current-time curves and ECL spectra of BB NRs-modified and Ru(bpy)₃²⁺-modified GCE, respectively.

In Table 3, relative ECL efficiencies of BB NRs/GCE and Ru(bpy)₃²⁺/GCE are illustrated.

TABLE 3

| System | I/10⁵ | Q | Φ_{ECL} |
|---|---|---|---|
| BB NRs-TPrA | 41.7 | 0.00421 | 56.7% |
| Ru(bpy)₃²⁺-TPrA | 115 | 0.00659 | 100% |
| BB NRS-K₂S₂O₈ | 34.7 | 0.0500 | 24.6% |
| Ru(bpy)₃²⁺-K₂S₂O₈ | 107 | 0.0344 | 100% |

Note that φ_{ECL} is calculated by the following equation (1).

$${Φ}_{ECL} = {Φ}_{ECL}^{°} ( I / Q ) / ( I ° / Q ° )$$

Here, φ and φ°, I and I°, and Q and Q° respectively indicate ECL efficiencies, ECL intensity integrals (integrated ECL spectrum vs. wavelength), and charge consumption (integrated CV curve vs. time) of a sample and a standard.

As seen from FIG. 12A to FIG. 12D and Table 3, the BB NRs of the present invention are more excellent in ECL efficiency than conventional fluorescence-based Pdots (Z. Wang et al., Adv. Funct. Mater. 2020, 30, 2000220; Y. Feng et al., Anal. Chem. 2016, 88, 845; Z. Wang et al., J. Phys. Chem. Lett. 2018, 9, 5296) and conventional fluorescence-based TADF Pdots (C. Wang et al., Anal. Chem. 2022, 94, 15695) in the presence of a coreactant having the same or a lower concentration. This is because the hot exciton mechanism gives BB NRs a high radiative transition rate and high utilization efficiency of excitons.

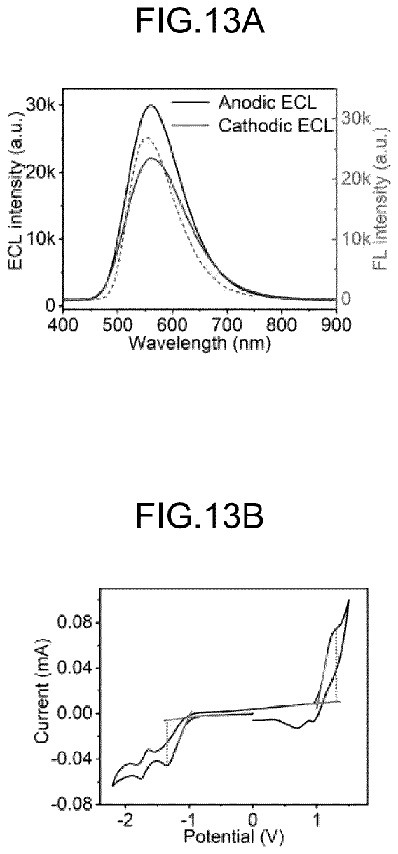

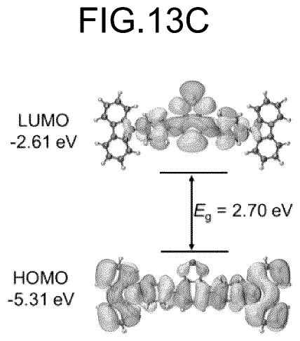

Furthermore, the ECL attribution of the BB NRs was analyzed. The results of the analysis are illustrated in FIG. 13A to FIG. 13C. FIG. 13A to FIG. 13C are diagrams illustrating the analysis results of ECL attribution of the BB NRs produced in Example 1 of the present invention. Here, FIG. 13A is a diagram illustrating anodic and cathodic ECL and FL spectra of the BB NRs, and FIG. 13B is a diagram illustrating a CV curve of a glass carbon electrode modified with the BB NRs. FIG. 13C is a schematic diagram illustrating a band gap between the lowest unoccupied molecular orbital (LUMO) and the highest occupied molecular orbital (HOMO) of the BCzP-BT molecule.

First, anodic and cathodic ECL and FL spectra of the BB NRs were tested. The test results are illustrated in FIG. 13A. Note that specific test conditions for the anodic and cathodic ECL spectra are the same as those for the anodic and cathodic ECL experiments described above.

FIG. 13A is a diagram illustrating the anodic and cathodic ECL and FL spectra of the BB NRs. As seen from FIG. 13A, the anodic and cathodic ECL emission peaks of the BB NRs matched well with the FL emission peaks thereof and all the peaks were at 560 nm, which indicated that the ECL and FL processes of the BB NRs had the same excited state. These excited states are generated by a band gap transition.

Furthermore, a CV curve in an organic phase of the BB NRs was tested. The test results are illustrated in FIG. 13B.

Here, specific test conditions for the organic phase CV are as follows.
Electrode material: BB NRs/GCE
Electrolyte: 0.1-M tetrabutylammonium chloride-containing tetrahydrofuran solution, nitrogen gas ventilation for 20 minutes
Test method: cyclic voltammetry
Maximum potential: +1.5 V
Minimum potential: −2.2 V
Scanning speed: 0.1 V/s

FIG. 13B is a diagram illustrating the CV curve of the glass carbon electrode modified with the BB NRs, and FIG. 13C is a schematic diagram illustrating the band gap between the lowest unoccupied molecular orbital (LUMO) and the highest occupied molecular orbital (HOMO) of the BCzP-BT molecule. As seen from FIG. 13B and FIG. 13C, the oxidation peak of the BB NRs corresponds to two carbazole oxidations, and the CV of BB NRs indicates an irreversible oxidation wave because carbazole easily electrochemically reacts to form a dimer, and a reduction peak thereof is generated by the reduction of benzothiadiazole and benzothiadiazole has two electron-withdrawing imino bonds (C═N), hence a CV curve thereof exhibits two successive reduction peaks.

### Example 2: ECL Nanoprobe

1) BB NRs Probe Modified with Oligonucleotide
a. Production of ssDNA-BB NRs

First, 12 μL of polyethylene glycol (PEG, Mw=3350) and 12 μL of 1-M 4-(2-hydroxyethyl)−1-piperazineethanesulfonic acid (HEPES) were sequentially added to 600 μL of BB NRs, and then the pH of the resulting solution was adjusted to 7.4 with 0.1-M NaOH, and then single-stranded DNA (ssDNA-BHQ2, 100 μM) modified with 40 μL of a black hole quencher 2 (BHQ2) and 24 μL of just-prepared EDC (10 mg/mL) were added to the solution. The resulting solution was stirred at room temperature for 24 hours in a dark place, and then free ssDNA-BHQ2 was removed by centrifugation by ultrafiltration to obtain ssDNA-BB NRs. The ssDNA-BB NRs were diluted so as to have the same absorbance at 410 nm as that of the BB NRs, and stored away from light.

b. Characteristics Analysis of ssDNA-BB NRs

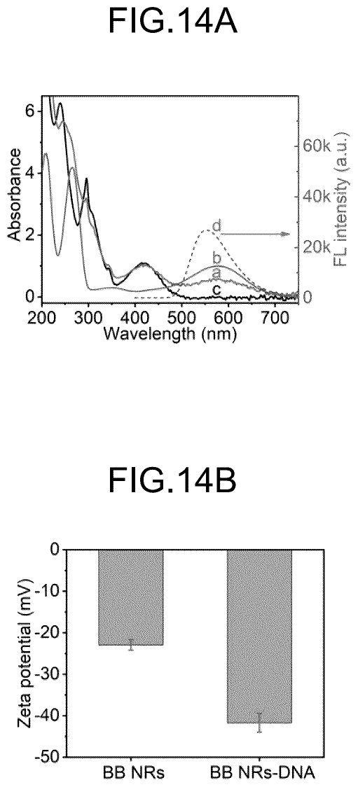

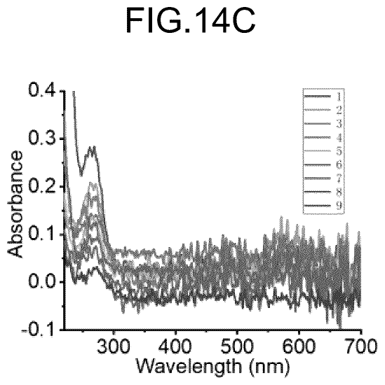

The ultraviolet absorption spectrum and Zeta potential of the ssDNA-BB NRs were measured. The results are illustrated in FIG. 14A to FIG. 14C. FIG. 14A to FIG. 14C are diagrams illustrating analysis results of characteristics of the ssDNA-BB NRs probe produced in Example 2 of the present invention. Here, FIG. 14A is a diagram illustrating the ultraviolet absorption and the FL spectra of the ssDNA-BB NRs probe. Curves a, b, c, and d in the figure indicate the ultraviolet absorption spectra of the ssDNA-BB NRs, the ssDNA, and the BB NRs, and the FL spectrum of the BB NRs, respectively. FIG. 14B is a diagram illustrating Zeta potential analysis results. FIG. 14C is a diagram illustrating ultraviolet analysis results of a solution ultrafiltrated each time during the ssDNA-BB NRs probe production process. Numbers 1 to 9 in the figure sequentially indicate the first to ninth filtrates.

As seen from the curve a in FIG. 14A, the ultraviolet (UV-vis) absorption of the ssDNA-BB NRs not only has a BB NRs absorption peak at 410 nm, but also has an ssDNA absorption peak at 260 nm and a BHQ2 absorption peak at 580 nm. Furthermore, as seen from FIG. 14A, a broad absorption band of the BHQ2 overlaps the FL emission peak of the BB NRs (the curve d), which indicated that the BHQ2 was capable of quenching the emission of the BB NRs.

As seen from FIG. 14B, the ssDNA-BB NRs have a more negative zeta potential than the BB NRs because the Zeta potential is given to the ssDNA-BB NRs. These results indicated that the ssDNA-BB NRs probe was successfully produced.

When the ssDNABB NRs were purified by ultrafiltration, a filtrate was collected each time and ultraviolet thereof was measured. As seen from FIG. 14C, the absorption peaks of DNA and BHQ2 in the solution gradually decreased with an increase in the number of times of the ultrafiltration, and the results indicated that free ssDNA-BHQ2 was hardly contained in the produced ssDNA-BB NRs probe solution.

4) ECL Switch Based on ssDNA-BB NRs Nanoprobe
a. Principle of Signal Switch

The ssDNA-BB NRs nanoprobe will be described as a reporter probe. As illustrated in FIG. 2, an end of ssDNA is modified with BHQ, hence BB NRs are in the ECL OFF state. The ssDNA-BB NRs nanoprobe was dropped directly onto a surface of an ITO electrode to construct an ECL sensor interface. In the case where a Cas12a enzyme is not activated when the reaction solution is dropped onto the sensor interface, the BHQ is bound to surfaces of the BB NRs via the ssDNA and the sensor electrode indicates a low ECL intensity (ECL OFF). On the contrary, in the case where a target molecule (a target nucleic acid) is present and the Cas12a enzyme is activated, the cleavage of the ssDNA occurs, whereby the BHQ is separated from the surfaces of the BB NRs, the ECL of the BB Nrs is recovered, and a strong ECL signal is generated in the sensor interface (ECL ON).

b. Verification of Signal Switch

First, a switch response of the Cas12a enzyme to the ssDNA-BB NRs nanoprobe was analyzed by fluorescence.

Test conditions for a Cas enzyme-fluorescence test are as follows.
Detector: fluorescence excitation detector
Excitation wavelength: 365 nm
Emission wavelength range: 400 to 700 nm
PMT: 800 V
Before reaction: 50 μL of ssDNA-BB NRs+50 μL of water
After reaction: 50 μL of ssDNA-BB NRs+50 μL of Cas12a system
Cas12a system: 2.5 μL of Cas12a (1 μM)+5 μL of crRNA (1 μM)+5 μL of NE buffer (10×)+32.5 μL of DEPC water+5 μL of HPV16 dsDNA (500 nM)

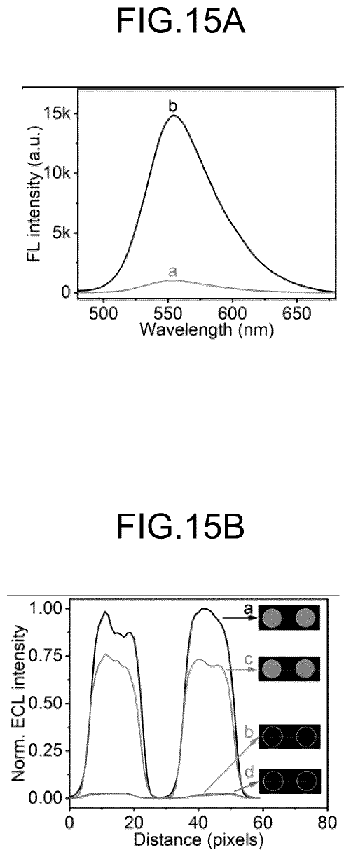

The results of the fluorescence test are illustrated in FIG. 15A and FIG. 15B. FIG. 15A and FIG. 15B are diagrams illustrating feasibility analysis results of the signal switch of the ssDNA-BB NRs probe produced in Example 2 of the present invention. Here, FIG. 15A is a diagram illustrating fluorescence analysis results before (curve a) and after (curve b) the reaction of the ssDNA-BB NRs and the Cas12a system. FIG. 15B is a diagram illustrating analysis results of normalized ECL imaging intensities of different ITO electrodes. Here, the curve a indicates an electrode modified with the BB NRs, the curve b indicates an electrode modified with the ssDNA-BB NRs, the curve c indicates a result of adding 1 nM target molecules to an electrode modified with the ssDNA-BB NRs+the Cas12a+crRNA system, and the curve d indicates a result of not adding any target molecules an electrode modified with the ssDNA-BB NRs+the Cas12a+the crRNA.

FIG. 15A is a diagram illustrating the fluorescence analysis results before (the curve a) and after (the curve b) the reaction of the ssDNA-BB NRs and the Cas12a system. As illustrated in FIG. 15A, the fluorescence of the ssDNA BB NRs was greatly recovered after the Cas12a enzyme was activated with a 1-nM target molecule (the curve b).

FIG. 15B is a diagram illustrating the analysis results of normalized ECL imaging intensities of the different ITO electrodes. As illustrated in FIG. 15B, compared to the sensor electrode (the curve a) modified with the BB NRs, the ECL imaging signal (the curve b) was hardly observed in the sensor interface modified with the ssDNA-BB NRs, which indicated that the sensor interface was in the ECL OFF state.

After the 1-nM target molecule was added, the Cas12a enzyme was activated, the cleavage of BHQ-ssDNA on the BB NRs occurred, an ECL ON response was generated, and consequently the ECL intensity of the sensor electrode was recovered up to 75% (the curve c). The results indicated the feasibility of an ECL switch system based on the ssDNA-BB NRs nanoprobe.

In addition, as seen from FIG. 15B, the binding of the DNA-BHQ2 to the BB NRs resulted in 95.6% quenching (the curves a, b) of the ECL emission of the BB NRs.

### Example 3: Method for Electrochemiluminescence Detection of HPV Gene Based on CRISPR/Cas12a Conversion Catalyst

ECL Imaging Detection of Sensor Array Constructed Based on ssDNA-BB NRs
a. Design of Detection

The detection method is constructed based on the “ECL switch of ssDNA-BB NRs nanoprobe” (FIG. 2) and used to detect HPV genes.

b. Production of Detection Chip and Detection by Detection Chip: A Specific Detection Flow is Illustrated in FIG. 3.

Specific operational steps are as follows.

Step 1: Indium tin oxide (ITO) glass was ultrasonically cleaned with toluene, acetone, ethanol, and ultrapure water for 5 minutes each, and then plated with a 5-nanometer thick chromium layer and a 50-nanometer thick gold layer by an electron beam evaporation sputtering apparatus to obtain an Au/ITO electrode.

Step 2: A porous seal (2 mm in diameter, 1 mm in depth, 4×7 well array) is attached to a surface of the Au/ITO electrode.

Step 3: 3 μL of the ssDNA-BB NRs was dropped into each well and dried at 37° C. for 30 minutes, and then each well was carefully washed with diethylpyrocarbonate (DEPC) water.

Step 4: 0.15 μL of LbCas12a (1 μM), 0.3 μL of crRNA (750 nM), 0.3 μL of a NE buffer (10×), and 1.95 μL of DEPC water were allowed to react at 37° C. for 10 minutes, and then 0.3 μL of HPV16 dsDNA or a sample having different concentrations was added thereto, and the resulting mixed solution was quickly dropped into each well and incubated at 37° C. for 40 minutes.

Step 5: Each well on the chip was washed three times with DEPC water to remove released BHQ2, and then the resulting was dried at 37° C. for 5 minutes. After that, the resulting was used for ECL imaging detection.

Step 6: ECL imaging detection was performed in a dark box by using an Au/ITO electrode as a working electrode, an Ag/AgCL electrode as a reference electrode, a platinum wire electrode as a counter electrode, an electron multiplying charge coupled device (EMCCD, iXon Ultra, Andor, UK) as a signal read-out device, and a 0.1-M pH 7.4 phosphate buffer solution of 5-mM tri-n-propylamine (TPrA) as an electrolyte. Cyclic voltammetry scanning was performed at 0 to +1.0 V, in which the scanning speed was 0.1 V/s and the exposure time was 20 seconds.

c. Optimization of ECL Detection Conditions

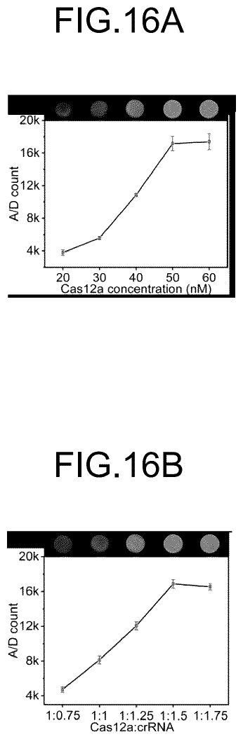

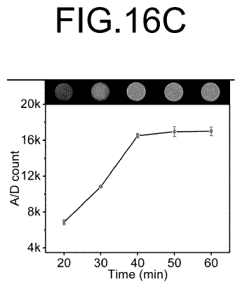

First, ECL detection according to the present invention was performed under different detection conditions. The results of the detection are illustrated in FIG. 16A to FIG. 16C. FIG. 16A to FIG. 16C are diagrams illustrating the detection results of the ECL detection performed under the different detection conditions in Example 3 of the present invention. Here, in FIG. 16A, detection results at different Cas12a concentrations are illustrated. In FIG. 16B, detection results at different usage ratios of Cas12a to crRNA are illustrated. In FIG. 16C, detection results for different incubation times on the chip are illustrated.

Specifically, as illustrated in FIG. 16A to FIG. 16C, the ECL detection was performed at different Cas12a concentrations, and different usage ratios of Cas12a to crRNA for different reaction times on the chip, and ECL imaging measurements were performed with the electrolyte for the chip under the different reaction conditions.

In FIG. 16A, detection results at different Cas12a concentrations are illustrated. In FIG. 16B, detection results at different usage ratios of Cas12a to crRNA are illustrated. In FIG. 16C, detection results at different incubation times on the chip are illustrated.

In the detection, conditions are selected so that an ECL signal reaches the maximum platform period, and are set as optimal detection conditions. As illustrated in FIG. 16A to FIG. 16C, the optimal conditions for ECL detection according to the present invention are a Cas12a concentration of 50 Nm, a ratio of Cas12a:crRNA of 1:1.5, and an incubation time of 40 minutes.

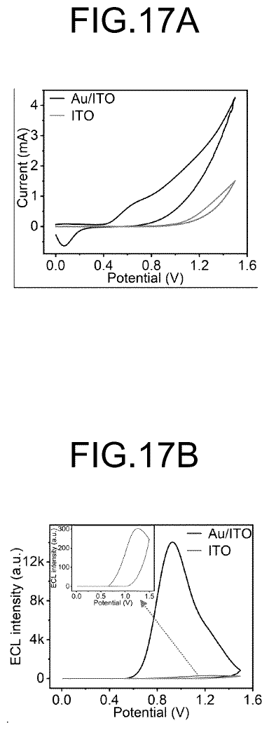

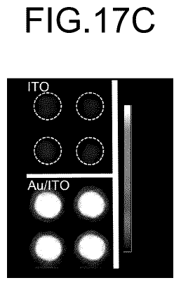

Next, ECL images of ITO and Au/ITO electrodes modified with BB NR in 5-mM TPrA-containing 0.1 M, pH 7.4 PBS were compared by ImageJ software. The results are illustrated in FIG. 17A to FIG. 17C. FIG. 17A to FIG. 17C are diagrams illustrating results of ECL imaging using the ITO and Au/ITO electrodes in Example 3 of the present invention. Here, FIG. 17A, FIG. 17B, and FIG. 17C are CV curves, ECL curves, and ECL images of the ITO and Au/ITO electrodes modified with BB NR, respectively.

As seen from FIG. 17A, FIG. 17B, and FIG. 17C, compared to the ITO modified with the BB NRs, the Au/ITO modified with the BB NRs exhibits a higher oxidation current, an oxidation peak shifted to 0.93 V, a 46-times stronger ECL response, and higher ECL-imaging quality. The reason for the differences is presumably that gold has a good catalytic ability for the oxidation of TPrA.

d. Detection Performance

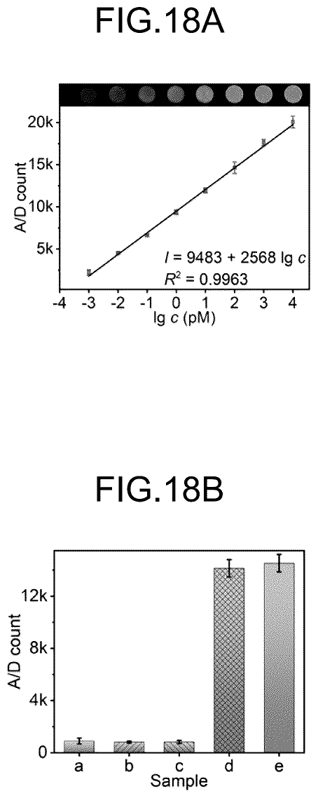

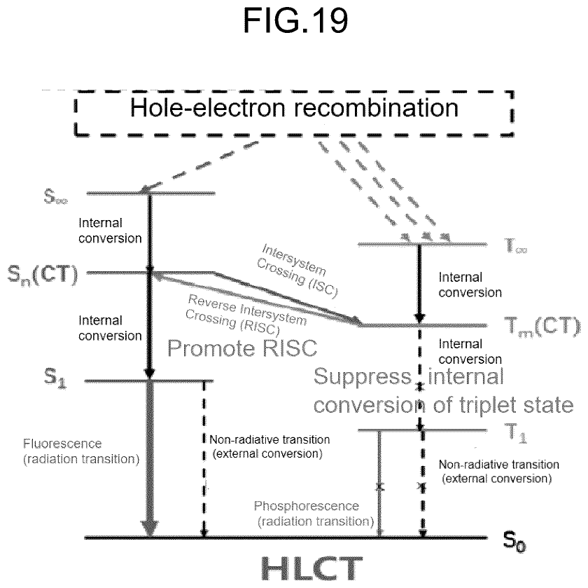

FIG. 18A and FIG. 18B are diagrams illustrating the results of ECL detection of the HPV16 DNA target nucleic acid in Example 3 of the present invention by using a sensor array. Here, in FIG. 18A, ECL imaging results and a linear curve obtained by detecting HPV16 DNA having a 1 fM to 10 nM concentration are illustrated. In FIG. 18B, analysis results of detection specificity of ECL detection for the HPV16 DNA. Here, a is 1-nM HPV18 DNA, b is 1-nM HPV31 DNA, c is 1-nM HPV58 DNA, d is 100-pM HPV16 DNA, and e is a mixed solution of 1-nM HPV18, 31, 58 DNA and 100-pM HPV16 DNA.

Detection Linearity and Sensitivity: ECL imaging detection was performed for a HPV16 DNA target molecule by a constructed sensor array under optimal detection conditions. The results are illustrated in FIG. 18A. In the upper part of FIG. 18A, the results of imaging detection for the HPV16 DNA having a concentration of 1 fM to 10 nM are illustrated. From the results, it was found that the intensity of ECL increased with an increase in HPV16 DNA concentration. When the logarithms of analog-to-digital conversion values (A/D count) of ECL intensity and HPV16 DNA concentrations were plotted, a satisfactory linear relationship within a range of 1 fM to 10 nM was observed. In the calculation based on 3S/N, the detection limit of the method for the HPV16 DNA is 0.6 fM.

Test for detection selectivity: In the present example, detection specificity for the target nucleic acid HPV16 DNA was tested in the presence of interfering DNA having a 10-fold higher concentration. The test results are illustrated in FIG. 18B. In FIG. 18B, the results of ECL detection for 100-pM HPV16 DNA (d) in the presence of 1-nM HPV18 DNA, 1 nM HPV31 DNA, or 1-nM HPV58 DNA alone (a, b, c) or in coexistence thereof (e) are illustrated. The results revealed that only a HPV16 DNA-containing sample solution had a clear ECL signal and the present method had satisfactory selectivity for the detection of HPV16 DNA.

### INDUSTRIAL AVAILABILITY

As seen from the above-described examples, by using BCzP-BT as a luminescent molecule, the present invention produced hot exciton organic nanorods (BB NRs) exhibiting sensitive annihilation ECL and the luminescence of a strong coreactant ECL for the first time. Because of the high Φ_{PL} owing to the locally-excited state and high exciton utilization efficiency owing to the charge transfer state, the hot exciton organic nanorods are superior in Φ_{ECL} to conventional fluorescent materials and thermally activated delayed fluorescent materials. Furthermore, prompt and high-throughput detection of HPV16 DNA with a simplified operating procedure was achieved by using the ECL nanoprobe of the present invention, identifying a target nucleic acid by the CRISPR/Cas enzyme system, and switching the signal from off to on. Thus, according to the present invention, a highly sensitive ECL detection platform for detecting HPV16 DNA can be constructed. The detection effect of the platform is similar to an ECL detection method using Ru(bpy)₃²⁺ as an ECL standard. Thus, the ECL detection method according to the present invention is a feasible and reliable ECL detection method.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A method for producing an electrochemiluminescence nanoprobe, the method comprising:
polymerizing a hot exciton organic luminescent molecule and a copolymer molecule to synthesize hot exciton nanoparticles; and
modifying the obtained hot exciton nanoparticles with an oligonucleotide chain modified with a quencher molecule to obtain modified hot exciton nanoparticles.

2. The method according to claim 1, wherein
the hot exciton organic luminescent molecule includes: a donor (D) structure selected from the group (a) consisting of the following chemical formulae (1); and an acceptor (A) structure selected from the group (b) of the following chemical formulae (2).

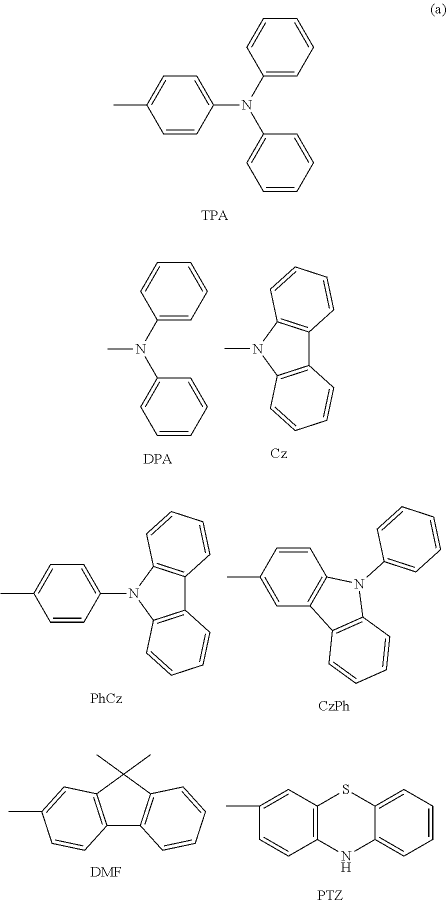

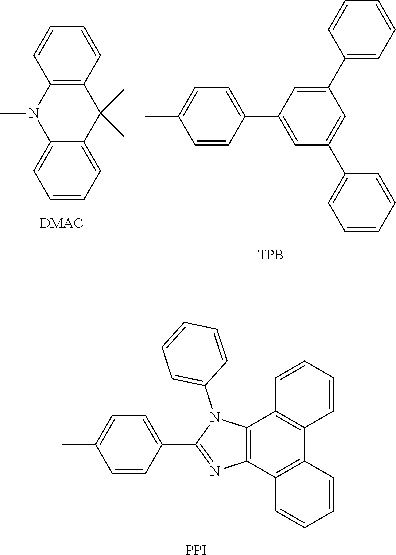

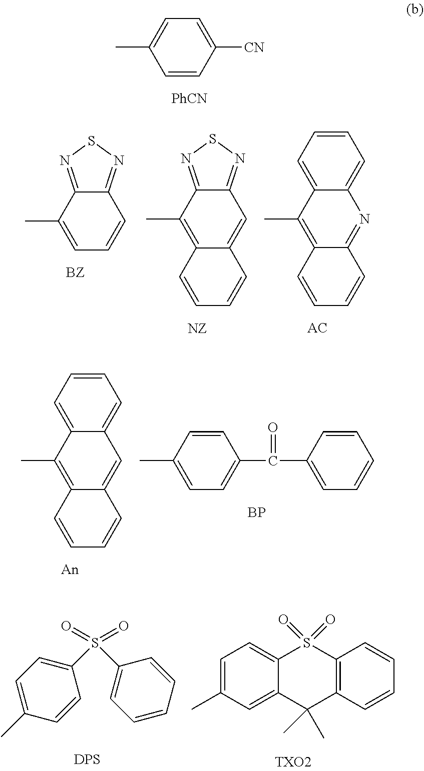

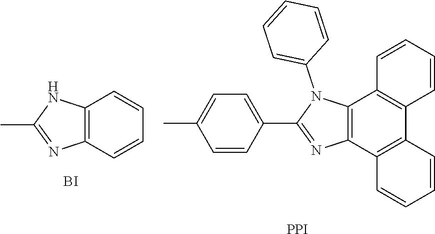

3. The method according to claim 2, wherein
the donor (D) is carbazole, and
the acceptor (A) is benzothiadiazole.

4. The method according to claim 3, wherein
the hot exciton organic luminescent molecule is BCzP-BT of the following chemical formula (3).

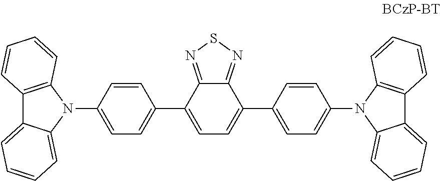 [SMILES: c1ccc2c(c1)c1ccccc1n2-c1ccc(-c2ccc(-c3ccc(-n4c5ccccc5c5ccccc54)cc3)c3nsnc23)cc1]

5. The method according to claim 1, wherein
the copolymer molecule is one of a polystyrene-polyacrylic acid block copolymer (PS-PAA), a polystyrene-maleic anhydride copolymer (PSMA), and a poly(isobutylene-alt-maleic anhydride) (PIMA), which are expressed by the following chemical formulae (4).

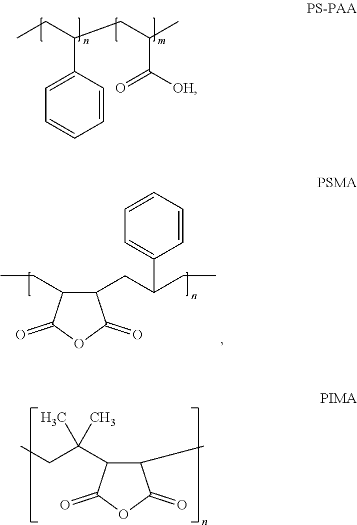

6. The method according to claim 5, wherein the hot exciton nanoparticles are synthesized from the hot exciton organic luminescent molecule and the copolymer molecule by a nano-coprecipitation method.

7. The method according to claim 1, wherein the hot exciton nanoparticles are one of nanoballs, nanotubes, nanorods, and nano-onions.

8. The method according to claim 1, wherein the quencher molecule is one of a black hole quencher, a dark quencher, and an amine reactive quencher.

9. An electrochemiluminescence nanoprobe, the electrochemiluminescence nanoprobe being hot exciton nanoparticles, the hot exciton nanoparticles being obtained by polymerizing a hot exciton organic luminescent molecule and a copolymer molecule and modified with an oligonucleotide chain modified with a quencher molecule.

10. An electrochemiluminescence sensor, the electrochemiluminescence sensor being a working electrode on which an electrochemiluminescence nanoprobe is dropped, the electrochemiluminescence nanoprobe being hot exciton nanoparticles, the hot exciton nanoparticles being obtained by polymerizing a hot exciton organic luminescent molecule and a copolymer molecule and modified with an oligonucleotide chain modified with a quencher molecule.

11. The electrochemiluminescence sensor according to claim 10, wherein the working electrode is one of a glass carbon electrode, an indium tin oxide electrode, and a screen printed electrode.

12. The electrochemiluminescence sensor according to claim 11, wherein the working electrode is a gold-indium tin oxide electrode.

13. An electrochemiluminescence detection method, the method using an electrochemiluminescence sensor, the electrochemiluminescence sensor being a working electrode onto which an electrochemiluminescence nanoprobe is dropped, the electrochemiluminescence nanoprobe being hot exciton nanoparticles, the hot exciton nanoparticles being obtained by polymerizing a hot exciton organic luminescent molecule and a copolymer molecule and modified with an oligonucleotide chain modified with a quencher molecule.

14. The electrochemiluminescence detection method according to claim 13, comprising:
adding a sample to be measured to a Cas enzyme catalyst system to obtain a sample reaction solution, the Cas enzyme catalyst system including a guide nucleic acid capable of binding to a target nucleic acid; and
adding the sample reaction solution to the electrochemiluminescence sensor, collecting an electrochemiluminescence signal, and analyzing the electrochemiluminescence signal.

15. The electrochemiluminescence detection method according to claim 14, wherein a concentration of Cas protein in a Cas enzyme in the sample reaction solution is 40 nM or higher.

16. The electrochemiluminescence detection method according to claim 14, wherein an amount of crRNA used is greater than an amount of the Cas protein.

17. The electrochemiluminescence detection method according to claim 13, wherein an incubation time for the sample reaction solution on the electrochemiluminescence sensor is 30 minutes or longer.

18. A kit for electrochemiluminescence detection, the kit comprising:
an electrochemiluminescence sensor being a working electrode onto which an electrochemiluminescence nanoprobe is dropped, the electrochemiluminescence nanoprobe being hot exciton nanoparticles, the hot exciton nanoparticles being obtained by polymerizing a hot exciton organic luminescent molecule and a copolymer molecule and modified with an oligonucleotide chain modified with a quencher molecule; and
a Cas enzyme-containing detection reagent.


SEQUENCE LISTING (10 sequences)

SEQ ID NO: 1 - DNA, 39 bp, human papillomavirus
  source 1..39: mol_type = genomic DNA
aatatgtcattatgtgctgccatatctacttcagaaact

SEQ ID NO: 2 - DNA, 39 bp, human papillomavirus
  source 1..39: mol_type = genomic DNA
agtttctgaagtagatatggcagcacataatgacatatt

SEQ ID NO: 3 - DNA, 39 bp, human papillomavirus
  source 1..39: mol_type = genomic DNA
aatttaacaatatgtgcttctacacagtctcctgtacct

SEQ ID NO: 4 - DNA, 39 bp, human papillomavirus
  source 1..39: mol_type = genomic DNA
aggtacaggagactgtgtagaagcacatattgttaaatt

SEQ ID NO: 5 - DNA, 39 bp, human papillomavirus
  source 1..39: mol_type = genomic DNA
ggtgaaccgaaaacggttggtatataaagcacatagtat

SEQ ID NO: 6 - DNA, 39 bp, human papillomavirus
  source 1..39: mol_type = genomic DNA
atactatgtgctttatataccaaccgttttcggttcacc

SEQ ID NO: 7 - DNA, 39 bp, human papillomavirus
  source 1..39: mol_type = genomic DNA
acagctagggcacacaatggtacatgtgcccataagcag

SEQ ID NO: 8 - DNA, 39 bp, human papillomavirus
  source 1..39: mol_type = genomic DNA
ctgcttatgggcacatgtaccattgtgtgccctagctgt

SEQ ID NO: 9 - DNA, 11 bp, synthetic construct
  source 1..11: mol_type = other DNA
tttttattttt

SEQ ID NO: 10 - RNA, 41 nt, synthetic construct
  source 1..41: mol_type = other RNA
taatttctactaagtgtagattgaagtagatatggcagcac